(19) United States
(12) Patent Application Publication
(10) Pub. No.: US 2005/0238594 A1
(43) Pub. Date: Oct. 27, 2005

(54) Block ethylenic copolymers comprising a vinyllactam block, cosmetic or pharmaceutical compositions comprising them and cosmetic use of these copolymers

(76) Inventor: Nathalie Mougin, Paris (FR)

Correspondence Address: Thomas L. Irving;FINNEGAN, HENDERSON, FARABOW,, GARRETT & DUNNER , L.L.P., 1300 I Street, N.W., Washington, DC, 20005-3315, US

(21) Appl. No.: 10/940,763

(22) Filed: Sep. 15, 2004

Related U.S. Application Data
(60) Provisional application No. 60/511,072, filed on Oct. 15, 2003.

(30) Foreign Application Priority Data
Sep. 15, 2003 (FR) 03 50537

(51) Int. Cl. A61K 7/42; A61K 7/06; A61K 7/11; C08F 26/08

(52) U.S. Cl. 424/59; 424/70.15; 526/264

(57) ABSTRACT

The present disclosure relates to linear block ethylenic copolymers that have reduced tack and thus do not leave a tacky feel, and to cosmetic and pharmaceutical compositions comprising the linear block ethylenic copolymer. Further disclosed herein is a cosmetic process for making up or caring for keratin materials, comprising the application to the keratin materials of such a composition, and also discloses herein are various cosmetic uses of such a composition. The tack of the copolymers disclosed herein is even reduced under conditions of high humidity, for example for RH values ranging from 50% to 100%.

This application claims benefit of U.S. Provisional Application No. 60/511,072, filed Oct. 15, 2003.

The present disclosure relates to novel polymers of specific structure, in particular to specific block ethylenic copolymers comprising a vinyllactam block. Also disclosed herein are compositions, such as cosmetic or pharmaceutical compositions, for instance compositions for hair, skin, and nails, comprising the block ethylenic polymer of specific structure. Still further disclosed herein are methods for cosmetic treatment of keratin materials, for example, the skin, the nails, and the hair, comprising applying these polymers, or compositions containing the polymers, to the hair.

The term “vinyllactam block” means a block comprising a lactam unit, for example, lactam derivatives. In general, a “vinyllactam block” is a block that can be prepared by polymerization of a monomer containing a lactam ring.

Many cosmetic compositions, for instance hair compositions, which can be in the form of aerosols (“sprays”), gels, mousses or shampoos, contain resins or polymers. These resins or polymers may be, for example, acrylic polymers with high glass transition temperatures (Tg), such as those described in document FR-A-2 439 798. Such polymers can provide, for instance, in terms of styling, hold of the hair, but they can have the drawback of being excessively brittle, which does not provide good hold of the hair over time.

In the case of varnishes, the existing polymers are not always resistant to impacts.

To solve the problems posed by these polymers, plasticizers are used in cosmetic compositions in order to reduce the glass transition temperature. However, when the plasticizers are used, the polymers have a tendency to exhibit “tacky” effects or, in the case of styling, do not allow “ultra-strong” fixing. In addition, once the hair is styled, numerous existing hairstyling polymers form white particles, which is unacceptable to the consumer, for instance, when the hair is brown and/or thick. Other drawbacks that can be presented by the polymers currently used in the art are their possible incompatibility with known aerosol propellants.

Polymers based on vinyllactams, for example on vinylpyrrolidone, and such as homopolyvinylpyrrolidone, are moreover also known in the cosmetics field. These polymers, however, have a high hygroscopicity, which can give them a highly tacky nature in the presence of ambient moisture.

Thus, for example, the document EP-A-1,002,811 from BASF describes water-soluble or water-dispersible grafted polymers obtained by free-radical polymerization of essentially acrylic monomers, and of a polymerizable prepolymer based on vinyllactam, for example on vinylpyrrolidone or vinylcaprolactam. These polymers can be used for instance, in hair compositions.

U.S. Pat. No. 6,193,961 from ISP describes a homogeneous terpolymer of N-vinyllactam, such as N-vinylpyrrolidone or N-vinylcaprolactam, of a dimethylaminoalkyl acrylate or of dimethylaminoalkylacrylamide and of a polysiloxane monomer. These terpolymers can be used in cosmetic and care compositions, for example cosmetic compositions such as styling gels or foams, or mousses. U.S. Pat. No. 6,193,961 cites numerous other patents mentioning the use of vinyllactam-based polymers in skincare and haircare compositions, such U.S. Pat. Nos. 3,914,403; 3,954,960; 4,039,734; 4,057,533; 4,210,161; 4,223,009; 4,586,518; 4,764,363; 4,834,968; 4,842,850; 4,902,499; 4,906,459; 4,923,694; 4,963,348; 5,011,895; 5,015,708; 5,126,124; 5,158,762; 5,275,809; 5,502,136; and the documents WO-A-91/15186, WO-A-91/15185, EP-A2-412 704, EP-A1-0 412 707 and JP-A-57 126 409. Further, in U.S. Pat. No. 6,193,961, it is also indicated that many patents, including U.S. Pat. Nos. 3,910,862; 4,923,694; 5,045,617; 5,321,110; 5,492,988 and 5,637,296, describe the use, for example, of an N-vinyllactam in the field of cosmetics and pharmaceutical products, such as in hair aerosols.

U.S. Pat. No. 3,954,960 relates to cosmetic and hair compositions containing, as film-forming resin, a quaternized copolymer of vinylpyrrolidone and of a copolymerizable vinyl monomer, i.e. a dialkylaminoalkyl (meth)acrylate. U.S. Pat. No. 3,914,403 relates to hair compositions containing a mixture of film-forming resins of an N-vinylpyrrolidone homopolymer or copolymer mixed with a quaternized copolymer of vinylpyrrolidone and of a vinyl monomer that is copolymerizable therewith, i.e. a dialkylaminoalkyl (meth)acrylate. U.S. Pat. No. 5,502,136 relates to a process for preparing copolymers of vinylpyrrolidone and of vinyl acetate via free-radical polymerization. WO-A-00/68282 relates to terpolymers based on vinylpyrrolidone (VP), dimethylaminopropylmethacrylamide (DMAPMA), and of the DMAPMA derivative quaternized with a C₈ to C₂₄ alkyl chain, and to hair and cosmetic compositions comprising them.

It is noted that, in these copolymers, fatty chains are inserted in order to reduce the tack and to increase the moisture resistance, but the range of properties of the polymers obtained may then become limited. Moreover, no value for the tack of these copolymers is mentioned.

The document by Matyjasezwski et al., Prepr. 38(1),1997, p. 695, describes copolymers containing an N-vinylpyrrolidone skeleton and PDMS grafts for hydrogels.

Patent WO 97/18247 from the same K. Matyjaszewski describes, on page 103, an example of a vinylpyrrolidone homopolymer. French patent FR-A-2,327,761 relates to cosmetic compositions comprising a polymer resulting from the polymerization, in the presence of cerium, of an unsaturated monomer onto a polyvinylpyrrolidone diol. A PVP-poly(lauryl methacrylate) polymer is mentioned. The use of a vinylpyrrolidone/glucosamine acrylate copolymer is also described. The polymers of this patent are very specific polymers, generally of branched structure. These polymers are said to give the hairstyle sheen and hold over time. However, they can also give the hairstyle a certain amount of rigidity, causing an unnatural appearance. They also have the drawback of being highly hygroscopic on account of the presence of unreacted hydroxyl units throughout the polymer chains.

There is thus a need for a polymer, which, when included in a composition, for example a cosmetic composition, such that this composition does not have the drawbacks, defects, limitations and disadvantages that can arise with the compositions of the prior art. There is, for instance, a need for a polymer and a composition containing it that has an optimum combination of rigidity and tack properties. Thus, a hair composition comprising the polymer should make it possible to obtain more hold, while at the same time maintaining a natural look. The polymer should for example, in such compositions, show good styling properties and, during disentangling, should not become powdery, i.e. should not form visible “flaking” residues. Moreover, the polymer should be compatible with aerosol propellent gases.

In the case of varnishing the nails, the production of a glossy film is desired, this film furthermore needing to be resistant to mechanical attack. The polymer contained in the formula should thus be capable of showing excellent resistance to mechanical abrasion.

In the case of a skin treatment, the makeup used, which includes the polymer, should adhere to the skin, without pulling on the skin, while at the same time being comfortable, i.e., not causing any “tautness.”

In all of the above cases and irrespective of the composition in which the polymer is used, it is necessary for this polymer to give a product with a non-tacky feel, for instance, as a function of the humidity, and such as under conditions of high humidity (for example for relative humidity (RH) values of from 50% to 100%). In other words, there is a need for a vinyllactam polymer that has little or no tack and, in any case, a reduced level of tack, in humidity, compared with the vinyllactam polymers of the prior art.

Accordingly, the present disclosure provides a polymer that satisfies, inter alia, at least one of the needs, criteria and requirements mentioned above and which solves at least one of the problems of the polymers of the prior art. Thus, the present disclosure relates to at least one linear block ethylenic copolymer comprising:
at least one block A that is obtained from monomers comprising a majority amount of an ethylenic monomer comprising a lactam ring chosen from monomers of formula (I): 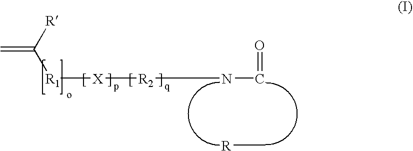
wherein:
   R is chosen from —(CH₂)ₙ— radicals, wherein at least one carbon atom is optionally replaced with a heteroatom chosen from nitrogen and oxygen atoms, wherein n is an integer ranging from from 3 to 12, and wherein at least one carbon atom is optionally substituted with at least one C₁ to C₆ alkyl group;
   R′ is chosen from an hydrogen atom and methyl radicals;
   R₁ and R₂, which may be identical or different, are chosen from linear, branched and cyclic alkylene and aralkylene radicals ranging from 1 to 22 carbon atoms, wherein at least one carbon atom is optionally replaced with a heteroatom chosen from oxygen and nitrogen atoms;
   X is chosen from —OCO—, —NHCO—, and —COO— radicals and —O— atoms;
   o, p and q represent, independently of each other, 0 or 1;
      and a minority amount of at least one non-hydrophilic monomer;
and at least one block B that is obtained from monomers not comprising an ethylenic monomer comprising a lactam ring of formula (I), or comprising a minor proportion thereof.

The majority amount (by weight) of the ethylenic monomer comprising a lactam ring is given relative to the total weight of the monomers from which the block A is obtained. In other words, the amount in units prepared from an ethylenic monomer comprising a lactam ring of block A is the majority amount by weight. This amount by weight, or weight content is given relative to the total weight of block A.

For instance, in one aspect of the present disclosure, in formula (I), o=0, p=1, q=1, R₂ is —CH₂CH₂—, X is chosen from COO and CONH radicals, and R is chosen from (CH₂)₃, (CH₂)₅, and CH₂CH₂NH radicals.

For example, the monomer of formula (I) may be chosen from pyrrilidinoethyl methacrylate or acrylate, and a ureidoethyl acrylate or methacrylate.

In one embodiment, the block A is obtained from monomers comprising a majority amount of a vinyllactam chosen from monomers of formula (II): 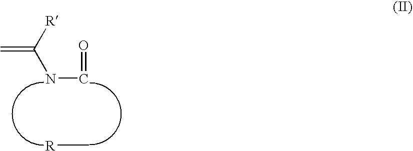
wherein R and R′ are as defined above.

For instance, in formulae (I) and (II) above, R₁ may be chosen from —(CH₂)ₙ—wherein n is an integer ranging from 3 to 5, or alternatively R is —CH₂—CH₂—NH—.

The N-vinyllactam of formula (II) may, for example, be chosen from N-vinylpyrrolidone (n=3), N-vinylpiperidinone (valerolactam) (n=4), N-vinylcaprolactam (n=5), N-vinylimidazolidinone wherein R is a —CH₂—CH₂—NH— group, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam and N-vinyl-7-ethyl-2-caprolactam.

In one embodiment of the present disclosure, the N-vinyllactams are chosen from N-vinylpyrrolidone, N-vinylcaprolactam and vinylimidazolidinone.

Another aspect of the present disclosure is cosmetic or pharmaceutical compositions comprising the linear block ethylenic copolymers. When the linear block ethylenic copolymers are incorporated into such compositions, the copolymers having the specific structure according to the present disclosure make it possible to obtain highly advantageous properties, or rather a combination of highly advantageous properties, which have not always been possible to obtain with the polymers of the prior art.

In general, the copolymers according to the present disclosure have, on account of their particular structure, reduced hygroscopicity when compared with the copolymers of the prior art containing vinyllactam units. The copolymers of the present disclosure have an optimum combination of rigidity and of non-tacky nature and they thus lead to compositions or systems having, for instance, at least one property chosen from enhanced mechanical strength, enhanced wear strength, enhanced hold over time, and reduced fragility, while at the same time not being tacky. The copolymers according to the present disclosure may thus be defined as tack-free polymers, which are, for instance, film-forming polymers.

Thus, when the copolymers as disclosed herein are used in compositions for treating the hair, such as lacquers, they can give greater hold over time. They can be less fragile than a standard lacquer and at the same time not be tacky. The phenomenon of powdering or flaking on the hair, which can be observed during disentangling with the compositions of the prior art, can thus be avoided.

In the case of nail varnishes, the formula comprising the copolymer according to the present disclosure can have greater wear strength and is not tacky, while at the same time adhering to the nail. As a result, the loss of gloss, i.e. the matting of the film by mechanical marking or by marking with dust which can take place with tacky films of the prior art, does not take place with the varnishes and films comprising the copolymer as disclosed herein. Without being bound by theory, the reason for this is that the absence of tack from these copolymers has the effect that they are not mechanically marked, thus they do not retain dust and they therefore do not undergo changes in or loss of gloss.

The varnishes comprising the copolymer according to the present disclosure give films that rarely or never chip, for instance by virtue of the presence in the copolymer as disclosed herein of a block A that is obtained from monomers comprising, besides the at least one ethylenic monomer comprising a lactam ring, such as a vinyllactam, present in a majority amount, at least one other minority (i.e., present in a minor amount) monomer which, specifically, are not hydrophilic. This particular structure of the copolymer of the present disclosure and, for example, of the block A, can prevent the films from chipping, but at the same time, and surprisingly, does not lead to a tacky film.

In makeup products, for instance lipsticks or foundations, the makeup can show good staying power on the lips or the skin, without giving a tacky sensation.

The present disclosure also relates to a cosmetic process for making up or caring for keratin materials, comprising the application to the keratin materials of a composition comprising the copolymer as described herein.

The present disclosure further relates to the use of the copolymers as disclosed herein to improve the styling power and/or the hairstyle hold, without tack, such as without a tacky feel, of a hair lacquer; the use of the copolymers as disclosed herein to improve the adhesion and the wear resistance, without tack, such as without a tacky feel, of a nail varnish; the use of the copolymers as disclosed herein to improve the staying power of a makeup composition; the use of the copolymers as disclosed herein to reduce the tack, for instance the tacky feel, of a cosmetic composition, such as under conditions of high humidity, for example for RH values of from 50% to 100%; and, finally, the use of the copolymers as disclosed herein in a cosmetic composition such as a care or makeup composition for masking wrinkles, which gives the skin a smoothened appearance, without tautness.

The copolymers according to the present disclosure thus provide a solution to at least some of the problems posed by the polymers of the prior art.

The unexpected advantageous properties of the specific copolymers of the present disclosure, which are fundamentally linear copolymers, are believed to arise, for example, from the specific nature of the blocks of which they are made, such as from the block A comprising a majority amount of ethylenic monomer comprising a lactam ring, such as a vinyllactam.

It appears that by virtue of the presence in the block A of at least one other non-hydrophilic monomers, the copolymers as disclosed herein, surprisingly, are of reduced hygroscopicity when compared with the vinyllactam-based polymers of the prior art. For example, the copolymers of the present disclosure have greatly reduced hygroscopicity when compared with the vinyllactam homopolymers of the prior art.

Moreover, the copolymers of the present disclosure are also, surprisingly, of reduced hygroscopicity when compared with the copolymers of the prior art represented, for example, by French Patent No. FR-A-2,327,761, which has a block structure formed firstly of blocks comprising vinyllactam homopolymers (for example of polyvinylpyrrolidone, PVP) and secondly of blocks prepared from non-hydrophilic monomers, such as methyl methacrylate.

In point of fact, it appears, surprisingly, that the incorporation of a non-hydrophilic monomer directly within the block of ethylenic monomer comprising a lactam ring, such as a vinyllactam, which is, as disclosed herein, in the form of an alternating gradient copolymer, or a random copolymer of the ethylenic monomer comprising a lactam ring and of the non-hydrophilic monomer, leads to a significant reduction in the hygroscopicity when compared with a copolymer in which the non-hydrophilic monomer is incorporated in the form of blocks in the copolymer that also comprise polyvinyllactam homopolymer blocks.

The copolymer according to the present disclosure also comprises, besides the at least one block A, obtained from a majority amount of an ethylenic monomer comprising a lactam ring, such as a vinyllactam, another block B, which is free of ethylenic monomers comprising a lactam ring such as a vinyllactam, or wherein if present, the ethylenic monomer comprising a lactam ring is present in a minority amount. The copolymers according to the present disclosure also show, as a result, a reduction in their hygroscopic and tacky nature when compared with copolymers of the prior art that are random copolymers of vinyllactam, for example of vinylpyrrolidone, and of a hydrophobic monomer, and which therefore do not comprise the block B according to the present disclosure.

Such random copolymers of the prior art, which are described, for example, in WO-A-00/68282 mentioned above, do not make it possible to obtain a reduction in the tack that is as great as that obtained with the copolymers as disclosed herein, and, in addition, they can show only a limited number of properties, which are reduced due to the fact of using a very specific non-hydrophilic or hydrophobic monomer. This limits the range of their possible applications. In contrast, the copolymers according to the present disclosure can have very varied structures on account of their block structure with a block B and can thus lead to properties that can be adapted according to the application of the copolymer.

The block B may be chosen as desired to give the copolymer as disclosed herein all the intended desirable properties—besides the non-tacky nature—that correspond to the specific applications. For example, the block B may be chosen so as to control the flexibility of the copolymer and to obtain, surprisingly, copolymers that are not fragile and brittle, and that give chip-free films, while at the same time not being tacky.

There is a need in the art for a copolymer that makes it possible to achieve a reduction in the hygroscopicity and the tacky nature of the copolymer. The specific structure of the copolymer of the as disclosed herein, i.e,
at least one block (A) comprising in a majority amount an ethylenic monomer comprising a lactam ring such as a vinyllactam;
wherein the at least one other monomer (present in a minority amount) of this block (A) is non-hydrophilic; and
at least one other block (B) having a structure different from the block (A) and being free of or comprising in a minor amount an ethylenic monomer comprising a lactam ring, such as an N-vinyllactam,
   leads to optimization of its properties, which can lead to a perfect equilibrium between, for example, the mechanical strength and non-tack properties.

Without wishing to be bound by theory, the advantageous properties of the copolymer according to the present disclosure are thought to arise from the fact that the nature of the blocks is chosen so as to promote the separation of the phases from each other and thus, inter alia, to give optimum control of the hygroscopicity and thus of the tack of the copolymer. Moreover, the fact that the polymer is linear entails a much simpler and much more controlled synthesis, which can make it possible to predict the structure of the polymers obtained, and thus to optimize the final properties of the polymers.

The copolymers according to the present disclosure are block copolymers. This term generally means that the copolymers comprise blocks that are covalently attached to each other. In addition, two successive blocks are of different nature. In contrast, two non-successive blocks A or B may be of the same nature.

As disclosed herein, each block A comprises a copolymer of the ethylenic monomer comprising a lactam ring, and of at least one non-hydrophilic monomer. Each block B comprises a homopolymer or a copolymer. The copolymer comprising block A and optionally block B may be chosen from random copolymers, alternating copolymers, and gradient copolymers.

The polymer may also comprise another block C other than the blocks A and B and optionally yet other blocks, for example a block D other than A, B and C. The polymer according to the present disclosure may thus be chosen from diblock copolymers of AB type, and triblock copolymers of ABA, BAB, ABC and ACB type, wherein C is other than A or B. The polymer according to the present disclosure may also be chosen from multiblock copolymers comprising more than three identical or different blocks: (AB)ₙ, (ABA)ₙ, (BAB)ₙ, (ABC)ₙ and (ACB)ₙ, wherein C is other than A or B, or multiblock copolymers comprising more than three different blocks of ABCD type.

In general, as has already been mentioned, the nature of the blocks is chosen so as to promote the phase separation between the blocks, since this is related to the properties. The nature and number of the blocks can be chosen by a person skilled in the art as a function of the desired properties within the limits of the conditions specified above for blocks A and B.

The copolymers of the present disclosure are defined as being ethylenic copolymers. This means that the monomers from which the blocks comprising the copolymer are derived are monomers comprising a carbon-carbon unsaturated double bond of ethylenic type.

In addition, the copolymer according to the present disclosure is a linear copolymer. Thus, the present disclosure does not cover copolymers having a non-linear structure, for example a branched, starburst, grafted or other structure. The linear nature of the copolymers as disclosed herein is directly related to the advantageous properties described above for the compositions comprising these copolymers.

In one aspect of the present disclosure, for example, the copolymer is a film-forming polymer, i.e. it is capable of, by itself, or in the presence of an auxiliary film-forming agent, at a temperature ranging from 20° C. to 30° C., forming a continuous film, capable of being viewed by the naked eye, and adhering to a keratin support.

According to the present disclosure, the copolymer comprises at least one block A that is obtained from monomers comprising a majority amount of an ethylenic monomer comprising a lactam ring of formula (I), for example a vinyllactam of formula (II).

As used herein, “majority amount” means that the monomers from which the at least one block A is obtained comprise an amount of ethylenic monomer comprising a lactam ring of formula (I), for example a vinyllactam of formula (II), greater than or equal to 50% by weight, relative to the total weight of the monomers from which the at least one block A is obtained, and lower than or equal to 99% by weight.

For example, the percentage of ethylenic monomers comprising a lactam ring of formula (I), such as a vinyllactam, among the monomers from which the at least one block A is prepared, may range from 50% to 99%, such as from 52% to 99%, and from 55% to 95% by weight, for instance, from 60% to 80% by weight, and from 65% to 75% by weight, and for example 70% by weight.

In other words, the amount (by weight) in units prepared from an ethylenic monomer comprising a lactam ring of block A is a majority amount, meaning greater than or equal to 50% by weight and lower than or equal to 99% by weight. These weight percentage amounts are given relative to the total weight of the at least one block A.

The major block A comprising a majority amount of ethylenic monomer comprising a lactam ring, such as a vinyllactam, may have any glass transition temperature Tg, but it generally has an overall “high” glass transition temperature of the block.

As used herein, the term “high glass transition temperature” generally means that the Tg ranges from 0 C to 250° C., for instance, from 0 C to 220° C., such as from 5 C to 200° C.

The block A may be hydrophilic or non-hydrophilic, i.e. hydrophobic. For example, depending on the hydrophobic unit comprised by the block A, it can remain water-soluble or, in contrast, can become insoluble.

A hydrophilic block may be defined as being a water-soluble or water-dispersible block. The definition of these terms is given later.

The ethylenic monomer comprising a lactam ring may be chosen from N-vinyllactams and derivatives thereof, which may comprise, for example, at least one C₁ to C₆ alkyl radical, such as a methyl, ethyl, n-propyl, isopropyl, n-butyl or sec-butyl radical.

In one embodiment of the present disclosure, the N-vinyllactam of formula (I) is chosen from pyrrilinoethyl acrylate and pyrrilidinoethyl methacrylate. In another embodiment, the N-vinyllactam is chosen from N-vinylpyrrolidone (n=3), N-vinylpiperidinone (valerolactam) (n=4), N-vinylcaprolactam (n=5), N-vinylimidazolidinone in which R is a —CH₂—CH₂-NH— group, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, and N-vinyl-7-ethyl-2-caprolactam.

In another embodiment of the present disclosure, the N-vinyllactams are chosen from N-vinylpyrrolidone, N-vinylcaprolactam and vinylimidazolidinone.

According to the present disclosure, the at least one block A, comprising a majority amount of an ethylenic monomer comprising a lactam ring, such as an N-vinyllactam, is a copolymer, further comprising, besides the ethylenic monomer comprising a lactam ring of formula (I), at least one other monomer, which may be identical or different, chosen from, according to the present disclosure, non-hydrophilic monomers, also known as hydrophobic monomers. These non-hydrophilic monomers are present in a minority amount.

By “minority amount” it is meant, for example, that the monomers from which block A is obtained, comprise generally an amount of the at least one non-hydrophilic monomer, of less than 50% by weight, relative to the total weight of the monomers from which the at least one block A is obtained.

In general, the amount of the non-hydrophilic or hydrophobic monomers in the monomers from which the block A is obtained is present in an amount ranging from 1% to less than 50% by weight, such as from 1% to 48%, and from 5% to 45% by weight, for instance, from 20% to 40%, and from 25% to 35% by weight, and for example 30% by weight.

In other words, the amount (by weight) in units obtained from a non-hydrophilic monomer of block A is a minority amount, meaning less than 50% by weight. These weight percentage amounts are given relative to the total weight of block A.

The monomers of the at least one block A, other than the ethylenic monomer comprising a lactam ring, are ethylenic monomers that are copolymerizable with the ethylenic monomer comprising a lactam ring, irrespective of the coefficient of reactivity thereof.

For example, the copolymer of block A may be a random copolymer, but it may also be chosen from alternating copolymers and gradient copolymers. The minority monomers, other than the ethylenic monomer comprising a lactam ring, from which the block A is obtained, may have a “high” or “low” glass transition temperature of the corresponding homopolymer. For example, these monomers may be chosen, for instance, from monomers for which the Tg of the corresponding homopolymer is “low”, i.e. generally less than or equal to 50° C., such as less than or equal to 20° C. and for example, less than or equal to 0° C. In one embodiment of the present disclosure, these monomers also have a Tg for the corresponding homopolymer that is greater than or equal to −150° C.

The block A, comprising a majority amount of ethylenic monomer comprising a lactam ring of formula (I) may be present in the copolymer as disclosed herein in an amount ranging from 1% to 99% by weight, relative to the total weight of the copolymer, such as from 10% to 95%, and for instance from 20% to 90% by weight, relative to the total weight of the copolymer.

The at least one block (B) is obtained from at least one ethylenic monomer generally chosen from: allylic monomers, acrylates, methacrylates, acrylamides, methacrylamides, vinyl monomers, and mixtures thereof, and optionally ethylenic monomers comprising a lactam ring, such as a vinyllactam, of formula (I) or (II), wherein the weight proportion of the monomers comprising a lactam ring of formula (I) or (II) is a minority proportion, i.e. less than 50% by weight, such as less than or equal to 45% by weight, for instance less than or equal to 40% by weight, and for example, less than or equal to 30% by weight.

The at least one block B may be hydrophobic or hydrophilic; for example, in one embodiment of the present disclosure, at least one of the blocks B is hydrophilic.

For further example, the at least one block B may have a low glass transition temperature Tg, such as less than or equal to 50° C., for instance, less than or equal to 20° C. and less than or equal to 0° C. The at least one block B may, for example, have a Tg value that is greater than or equal to −150° C.

In another embodiment of the present disclosure, the number-average molecular mass of each block, whether it is block A or block B, ranges from 2,000 to 1,000,000, for instance from 2,000 to 800,000, such as from 2,000 to 500,000. The number-average molecular mass of the overall copolymer (for example A-b-B) ranges from 4,000 to 1,000,000, such as from 4,000 to 800,000, and for instance from 4,000 to 500,000.

For example, the at least one block B may be present in the copolymer in an amount ranging from 1% to 99% by weight, relative to the total weight of the copolymer, for instance, from 5% to 90%, such as from 10% to 80% by weight, relative to the total weight of the final copolymer.

The copolymer according to the present disclosure will now be described in more detail.

Since the glass transition temperature Tg is used as a parameter for defining the blocks of the copolymer of the present disclosure, for example, block B of the copolymer of the present disclosure and, consequently, the copolymer as disclosed herein, it is important to point out that the glass transition temperatures of the blocks of the copolymers used in the present invention are measured by differential thermal analysis (DSC, “Differential Scanning Calorimetry”) for the dry polymer, at a heating rate of 10° C./minute.

Each block B of the copolymer according to the present disclosure is derived from one type of monomer or from several different types of monomer. This means that each block B may comprise a homopolymer or a copolymer, wherein the copolymer comprising the block may be chosen from random, alternating and gradient copolymers.

Each block A of the copolymer according to the present disclosure is chosen from alternating, random and gradient copolymers; this copolymer obviously comprising a majority proportion of ethylenic monomer comprising a lactam ring, such as a vinyllactam.

Each of the blocks A and B comprising the copolymer according to the present disclosure will now be described in detail.

According to the present disclosure, the block A, comprising a majority amount of ethylenic monomer comprising a lactam ring of formula (I), further comprises a minority amount of at least one non-hydrophilic monomer. The term “non-hydrophilic monomer” means that the homopolymer prepared from this monomer is not water-soluble or water-dispersible.

As used herein, a homopolymer is water-soluble if it is soluble in water to a proportion of at least 5% by weight at 25° C. As used herein, a homopolymer is water-dispersible if it forms, at a concentration of 5% by weight, at 25° C., a stable suspension of generally spherical fine particles. The mean size of the particles comprising the dispersion is less than 1 μm, for instance ranging from 5 nm to 400 nm, such as from 10 nm to 250 nm. The particle sizes are measured by light scattering.

The at least one non-hydrophilic minority monomer, other than the lactam of formula (I), from which the block A is prepared is for example, chosen from monomers for which the glass transition temperature of the corresponding homopolymer is low, such as less than or equal to 50° C., for instance, less than or equal to 20° C., and less than or equal to 0° C. In addition, the at least one non-hydrophilic monomerhas for example, a Tg value of greater than or equal to −150° C.

The at least one non-hydrophilic minority monomer of the block A may be chosen from, for example, the following monomers:
ethylenic hydrocarbons having from 2 to 10 carbon atoms, such as ethylene, isoprene or butadiene;
acrylates of formula CH₂═CHCOOR₃;
methacrylates of formula: 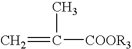
wherein R₃ is chosen from:
   linear and branched alkyl groups of 1 to 18 carbon atoms optionally interrupted with at least one hetero atom chosen from O, N, S and P atoms,
where the alkyl group is also optionally substituted with at least one substituent chosen from hydroxyl groups, halogen atoms, such as Cl, Br, I and F, and Si(R₄R₅) groups, wherein R₄ and R₅, which may be identical or different, are chosen from C₁ to C₆ alkyl groups and phenyl groups,
   C₃ to C₁₂ cycloalkyl groups, such as an isobornyl group,
   C₃ to C₂₀ aryl groups such as a phenyl group,
   C₄ to C₃₀ aralkyl groups (wherein the alkyl groups are C₁ to C₈) such as 2-phenylethyl or benzyl,
   4- to 12-membered heterocyclic groups comprising at least one heteroatom chosen from O, N and S, wherein the ring is aromatic or non-aromatic,
   heterocyclylalkyl groups, wherein the alkyls range from 1 to 4 carbon atoms, such as furfurylmethyl or tetrahydrofurfurylmethyl,
      wherein the cycloalkyl, aryl, aralkyl, heterocyclic and heterocyclylalkyl groups are optionally substituted with at least one substituent chosen from hydroxyl groups, halogen atoms and linear and branched alkyl groups comprising from 1 to 4 carbon atoms, in which the alkyl groups are optionally interrupted by at least one heteroatom chosen from O, N, S and P, the alkyl groups also being optionally substituted with at least one substituent chosen from hydroxyl groups, halogen atoms, such as Cl, Br, I and F, and Si(R₄R₅) groups, wherein R₄ and R₅, which may be identical or different, are chosen from C₁ to C₆ alkyl groups and phenyl groups. Among the examples of R₃ groups that may be used as disclosed herein, non-limiting mention may be made of methyl, ethyl, propyl, isobutyl, n-butyl, tert-butyl, hexyl, ethylhexyl, octyl, lauryl, isooctyl, isodecyl, dodecyl, cyclohexyl, t-butylcyclohexyl, t-butylbenzyl, isobornyl, phenyl, furfurylmethyl, tetrahydrofurfurylmethyl and 2-ethylperfluorohexyl groups. Still another example of an R₃ group for the acrylates includes —(OC₂H₄)ₘ—OR″, wherein m ranges from 5 to 150 and R″ is chosen from a hydrogen atom and C₆ to C₃₀ alkyl radicals, for example —POE-lauryl;
the (meth)acrylamides of formula: 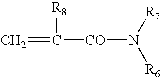
wherein R₈ is chosen from hydrogen atoms and methyl radicals, and R₇ and R₆, which may be identical or different, are chosen from
   hydrogen atoms and linear and branched alkyl groups of 1 to 18 carbon atoms, in which at least one heteroatom chosen from O, N, S and P, is optionally intercalated, the alkyl group also being optionally substituted with at least one substituent chosen from hydroxyl groups, halogen atoms, such as Cl, Br, I and F, and Si(R₄R₅) groups, wherein R₄ and R₅, which may be identical or different, are chosen from C₁ to C₆ alkyl groups and phenyl groups, non-limiting examples of these groups include methyl, ethyl, n-butyl, t-butyl, isopropyl, isohexyl, isooctyl and isononyl groups;
   C₃ to C₁₂ cycloalkyl groups, such as an isobornyl group,
   C₃ to C₂₀ aryl groups such as phenyl
   C₄ to C₃₀ aralkyl groups where the alkyl group ranges from C, to C₈ such as 2-phenylethyl or benzyl,
   4- to 12-membered heterocyclic groups comprising at least one heteroatom chosen from O, N and S, the ring being aromatic or non-aromatic,
   heterocyclylalkyl groups, wherein the alkyl group ranges from 1 to 4 carbon atoms), such as furfurylmethyl or tetrahydrofurfurylmethyl,
      wherein the said cycloalkyl, aryl, aralkyl, heterocyclic and heterocyclylalkyl groups are optionally substituted with at least one substituent chosen from hydroxyl groups, halogen atoms and linear and branched alkyl groups of 1 to 4 carbon atoms in which at least one heteroatom chosen from O, N, S and P is optionally intercalated, the alkyl groups also being optionally substituted with at least one substituent chosen from hydroxyl groups, halogen atoms, such as Cl, Br, I and F, and Si(R₄R₅) groups, wherein R₄ and R₅, which may be identical or different, are chosen from C₁ to C₆ alkyl groups and phenyl groups.

Among the examples of (meth)acrylamide monomers that may be used, non-limiting mention may be made of N-ethyl(meth)acrylamide, N-butylacrylamide, N-t-butylacrylamide, N-isopropylacrylamide, N,N-dibutylacrylamide, N-octylacrylamide, N-dodecylacrylamide and undecylacrylamide;
the allylic compounds of formula:
   CH₂═CH—CH₂—R₉ and CH₂═C(CH₃)—CH₂—R₉;
the vinyl compounds of formula:
   CH₂═CH—R₉,
wherein R₉ is a group chosen from:
   OR₁₀, in which R₁₀ is chosen from phenyl groups and C₁ to C₁₂ alkyl groups (the monomer is a vinyl ether or an allyl ether),
   OCOR₁₁, in which R₁₁ is chosen from:
      linear and branched alkyl groups of 2 to 12 carbon atoms (the monomer is a vinyl ester or an allylic ester),
      C₃ to C₁₂ cycloalkyl groups such as isobornyl or cyclohexyl,
      C₃ to C₂₀ aryl groups such as phenyl,
      C₄ to C₃₀ aralkyl groups (C₁ to C₈ alkyl group) such as 2-phenylethyl or benzyl,
      4- to 12-membered heterocyclic groups containing at least one heteroatom chosen from O, N and S, the ring being aromatic or non-aromatic,
      heterocyclylalkyl groups (1 to 4 C alkyl), such as furfurylmethyl or tetrahydrofurfurylmethyl,
         wherein the cycloalkyl, aryl, aralkyl, heterocyclic and heterocyclylalkyl groups are optionally substituted with at least one substituent chosen from hydroxyl groups, halogen atoms and linear and branched alkyl groups of 1 to 4 carbon atoms in which at least one heteroatom chosen from O, N, S and P is optionally intercalated, the alkyl groups also being optionally substituted with at least one substituent chosen from hydroxyl groups, halogen atoms, such as Cl, Br, I and F, and Si(R₄R₅) groups, in which R₄ and R₅, which may be identical or different, are chosen from C₁ to C₆ alkyl groups and phenyl groups.

Non-limiting examples of vinyl monomers include vinylcyclohexane and styrene.

Non-limiting examples of vinyl esters include: vinyl acetate, vinyl propionate, vinyl butyrate, vinyl ethylhexanoate, vinyl neononanoate and vinyl neododecanoate.

Among the vinyl ethers that may be used as disclosed herein, non-limiting examples include, ethyl vinyl ether and isobutyl vinyl ether;
(meth)acrylic or (meth)acrylamide and vinyl monomers containing a fluoro or perfluoro group, such as ethylperfluorooctyl methacrylate;
silicone (meth)acrylic or vinyl monomers, such as methacryloxypropyltris(trimethylsiloxy)silane, acryloxypropylpolydimethylsiloxane or silicone (meth)acrylamides.

Further examples of monomers that may be used include, for instance, those chosen from those for which the glass transition temperature Tg of the corresponding homopolymer is less than or equal to 50° C., such as: methyl acrylate (Tg=10° C.), ethyl acrylate (Tg=−24° C.), n-butyl acrylate (Tg=−54° C.), t-butyl acrylate (Tg=43° C.), 2-ethylhexyl acrylate (Tg=−50° C.), isobutyl acrylate (Tg=−24° C.), butyl methacrylate (Tg=20° C.), n-hexyl methacrylate, POE methacrylate (n=8 to 10) (Tg=−55° C.) and vinyl acetate (Tg=23° C.); as well as other monomers, such as methyl methacrylate (Tg=105° C.), ethyl methacrylate, isobutyl methacrylate, furfuryl acrylate, isobornyl acrylate, tert-butylcyclohexyl acrylate, styrene and vinylcyclohexane.

In one embodiment of the present disclosure, the non-hydrophilic minority monomers to make the block A are chosen from t-butyl acrylate, methyl acrylate, ethyl acrylate and butyl acrylate.

The copolymer according to the present disclosure also comprises at least one block B that may be obtained from monomers comprising no ethylenic monomer comprising a lactam ring, or comprising a minority proportion thereof.

The only limitation as regards the nature of the monomers that can create the at least one block B, is the limitation regarding the amount of ethylenic monomer comprising a lactam ring that may be used to prepare this block B. These monomers may be hydrophobic or hydrophilic. The terms “hydrophilic” and “non-hydrophilic” have already been defined above.

The at least one minority monomer, other than the ethylenic monomer comprising a lactam ring, from which the at least one block B is obtained may be chosen from a wide variety of monomers, the Tg values of these monomers may have any value, and these monomers may also be hydrophilic or hydrophobic.

In one embodiment of the present disclosure, the at least one block B has a low glass transition temperature Tg, for instance, less than or equal to 50° C., such as less than 20° C. and for example, less than 0° C.

The at least one minority monomer, from which the at least one block B may be prepared can be chosen, for example, from monomers for which the glass transition temperature of the corresponding homopolymer is low, such as less than or equal to 50° C., for instance, less than or equal to 20° C., and less than or equal to 0° C., and in one embodiment, for instance, from monomers for which the Tg of the corresponding homopolymer of greater than −150° C.

The monomers of the block B can also be chosen such that by combining, in the copolymer, monomers with a high Tg for the corresponding homopolymer, and monomers with a low Tg for the corresponding homopolymer, the overall Tg of the block is within the desired ranges. Thus, for example, by combining, to obtain the at least one block B, a monomer with a Tg for the corresponding homopolymer equal to 100° C. (for example methyl methacrylate) in a proportion of 35% by weight relative to the total weight of the monomers in the block B, and a monomer with a Tg for the corresponding homopolymer equal to −50° C., for example 2-ethylhexyl acrylate, in a proportion of 65% by weight, then the resulting block will have a Tg of −14° C., calculated to a first approximation by Fox's law: 1/Tg=sum (% Wa/Tga) wherein Wa=% by weight of the monomer a, and Tga=glass transition temperature of the homopolymer made from the monomer a.

These monomers may be chosen for example, from the non-hydrophilic monomers already mentioned above for the block A. However, unlike the block A, the monomers from which the block B may be prepared may also be chosen from hydrophilic monomers.

Non-limiting examples of hydrophilic monomers include ionic monomers such as cationic monomers, anionic monomers and betaines; nonionic monomers; and monomers that may be made hydrophilic after a hydrolysis, for example, of monomers of (meth)acrylic ester type made water-soluble by conversion to the corresponding carboxylic acid.

Non-limiting examples of cationic monomers that may be used as disclosed herein include:
2-vinylpyridine (Tg: 104° C.);
4-vinylpyridine (Tg: 142° C.);
dimethylaminoethyl (meth)acrylate;
diethylaminoethyl (meth)acrylate;
dimethylaminopropyl (meth)acrylamide; and
salified or quaternized forms thereof, whether they are salts of mineral acids, such as sulfuric acid or hydrochloric acid, or salts of organic acids.

These organic acids may comprise at least one group chosen from carboxylic, sulfonic and phosphonic acid group. They may be linear, branched or cyclic aliphatic acids or alternatively aromatic acids. These acids may also comprise at least one heteroatom chosen from O and N, for example in the form of hydroxyl groups.

A non-limiting example of an acid containing an alkyl group is acetic acid, CH₃COOH.

A non-limiting example of a polyacid is terephthalic acid.

Non-limiting examples of hydroxy acids include citric acid and tartaric acid.

Non-limiting examples of anionic monomers that may be used as disclosed herein include:
acrylic acid, methacrylic acid, crotonic acid, maleic anhydride, itaconic acid, fumaric acid and maleic acid;
styrenesulfonic acid, 2-acrylamido-2-methylpropanesulfonic acid, vinylsulfonic acid, vinylbenzoic acid, sulfopropyl methacrylate and vinylphosphoric acid, and the salts thereof.

The neutralizer may be a mineral base, such as LiOH, NaOH, KOH, Ca(OH)₂ or NH₄OH, or an organic base. It may also be a primary, secondary or tertiary alkylamine, such as triethylamine or butylamine. The primary, secondary or tertiary alkylamine may comprise nitrogens and/or oxygens and may thus comprise, for example, an alcohol functional group; it may thus be, for example, 2-amino-2-methylpropanol or triethanolamine.

Non-limiting examples of ionic monomers of betaine type that may be used as disclosed herein include:
ethylenic carboxybetaines or sulfobetaines obtained, for example, by quaternization of ethylenically unsaturated monomers comprising an amine functional group with carboxylic acid salts containing a labile halogen, for example sodium chloroacetate, or with cyclic sulfones, for example propane sulfone.

Non-limiting examples of nonionic monomers that may be used as disclosed herein include:
hydroxyalkyl (meth)acrylates or hydroxyalkyl(meth)acrylamides, the alkyl group of which comprises from 2 to 4 carbon atoms, for instance hydroxyethyl (meth)acrylate or hydroxypropyl (meth)acrylate;
(meth)acrylates or (meth)acrylamides of (C₁-C₄)alkoxy(C₁₋₄)alkyl, such as methoxyethyl, ethoxyethyl and methoxypropyl,
(meth)acrylates or (meth)acrylamides containing a group —(OC₂H₄)ₘ—OR′″, in which m is an integer ranging from 5 to 150 and R′″ is chosen from a hydrogen atom and C₁ to C₄ alkyl radicals, for example —POE-methoxy; —POE-OH;
vinyllactams, and
polysaccharide (meth)acrylates, for instance sucrose acrylate.

However, the vinyllactams may constitute only a minority proportion of the at least one block B.

The monomers that may be made hydrophilic as a result of a hydrolysis include, for example, monomers of (meth)acrylic ester type that can be hydrolyzed to acids, such as ethyl, tert-butyl or benzyl (meth)acrylate.

The hydrolysis will be performed once the polymer has been synthesized, under acidic conditions (such as sulfuric acid, hydrochloric acid or trifluoroacetic acid) or basic conditions (in the presence of alkaline-earth metal hydroxides such as sodium hydroxide or potassium hydroxide, alkali metal alkoxides such as potassium t-butoxide, or amines such as aqueous ammonia). The hydrolysis generally takes place at a temperature range from 5 C to 100° C., for instance from 15° C. and 80° C. The polymer will then be purified by repeated precipitations.

Among the hydrophilic monomers that may be used as disclosed herein, non-limiting mention may be made of:
acrylic acid, methacrylic acid, crotonic acid, maleic anhydride, vinylbenzoic acid, styrenesulfonic acid, 2-acrylamido-2-methylpropanesulfonic acid and vinylsulfonic acid,
vinyllactams, (in a minority proportion)
(meth)acrylamides and N-(C₁-C₄)alkyl(meth)acrylamides, for instance isobutylacrylamide,
polysaccharide (meth)acrylates, for instance sucrose acrylate, and
tert-butyl acrylate or ethyl acrylate followed by hydrolysis.

It should be noted that even though the copolymer comprises a hydrophilic block, the overall copolymer is not necessarily hydrophilic.

As previously disclosed hereinabove, the linear block ethylenic copolymers according to the present disclosure are chosen from:
diblock copolymers (AB);
triblock copolymers chosen from ABA, BAB, ABC and ACB), with C other than A or B,
multiblock copolymers comprising more than three blocks chosen from: (AB)ₙ, (ABA)ₙ, (BAB)ₙ, (ABC)ₙ and (ACB)ₙ, with C other than A or B, and multiblock copolymers comprising more than three different blocks, of ABCD type.

The copolymers according to the present disclosure may be prepared by anionic polymerization. However, the copolymers according to the present disclosure can also be obtained, for example, in a first mode by controlled free-radical polymerization and, for further example, the copolymers according to the may be obtained via a “ATRP” polymerization, such as the technique known as “Reverse ATRP”, or via the “RAFT” technique, but the polymers as disclosed herein may, for instance, according to a second mode, also be obtained via standard free-radical polymerization.

First Mode: Controlled Free-Radical Polymerization

The block copolymers according to the present disclosure can be obtained, for instance, by controlled free-radical polymerization, described for example in “New Method of Polymer Synthesis”, Blackie Academic Professional, London, 1995, Volume 2, page 1.

Controlled free-radical polymerization makes it possible to reduce the deactivation reactions of the growing free-radical species, such as the termination step, these being reactions which, in standard free-radical polymerization, can irreversibly and uncontrollably stop the growth of the polymer chain. In order to reduce the probability of the termination reactions, it has been proposed to temporarily and reversibly block the growing free-radical species, by forming “dormant” active species in the form of a bond with a low dissociation energy.

Thus, the polymerization may be performed according to the Atom Transfer Radical Polymerization (ATRP) technique, or by reaction with a nitroxide, or alternatively according to the “reversible addition-fragmentation chain transfer” (RAFT) technique, or, finally, by the “reverse ATRP” technique.

The atom transfer radical polymerization technique, also known as ATRP, consists in blocking the growing free-radical species in the form of a bond of carbon-halide type in the presence of a metal/ligand complex. This type of polymerization is reflected by control of the mass of the polymers formed and by a low dispersity index of the masses.

In general, the atom transfer radical polymerization is performed by polymerization of at least one free-radical-polymerizable monomer, in the presence of:
an initiator containing at least one transferable halogen atom;
a halogenated compound comprising a transition metal capable of participating in a reduction step with the initiator and a “dormant” polymer chain, this compound will be referred to as the “chain-transfer agent”; and
a ligand that may be chosen from compounds comprising a nitrogen (N), oxygen (O), phosphorus (P) or sulfur (S) atom, capable of coordinating via a sigma bond to the said compound comprising a transition metal, wherein the formation of direct bonds between the said compound comprising a transition metal and the polymer under formation is avoided.

The halogen atom may be, for example a chlorine or bromine atom.

This process is described, for instance, in WO 97/18247 and in the article by Matyjasezwski et al. published in JACS, 117, page 5614 (1995).

The technique of free-radical polymerization by reaction with a nitroxide comprise blocking the growing free-radical species in the form of a bond of C—O—NRₐR_{b} type, wherein Rₐ and R_{b}, which may be identical or different, are chosen from alkyl radicals comprising from 2 to 30 carbon atoms, or together forming, with the nitrogen atom, a ring comprising from 4 to 20 carbon atoms, for instance a 2,2,6,6-tetramethylpiperidyl ring. This polymerization technique is described for example in the articles “Living free radical polymerization: a unique technique for preparation of controlled macromolecular architectures”, C J Hawker; Chem. Res., 1997, 30, 373-382 and “Macromolecular engineering via living free radical polymerizations” published in Macromol. Chem. Phys. 1998, Vol. 199, pages 923-935, or alternatively in patent application WO-A-99/03894.

The RAFT (reversible addition-fragmentation chain transfer) polymerization technique comprises blocking the growing free-radical species in the form of a bond of carbomn-sulfur type. Dithio compounds such as dithioesters (—C(S)S—), such as dithiobenzoates, dithiocarbamates (—NC(S)S—) or dithiocarbonates (—OC(S)S—) (xanthates) are used to do this. These compounds make it possible to control the chain growth of a wide range of monomers. However, dithioesters inhibit the polymerization of vinyl esters, while dithiocarbamates are very weakly active towards methacrylates, which limits the application of these compounds to a certain extent. This technique is described for example in patent application WO-A-98/58974 from Rhodia and in the article “A more versatile route to block copolymers and other polymers of complex architecture by living radical polymerization: the RAFT process”, published in Macromolecules, 1999, Volume 32, pages 2071-2074. The already mentioned patent application WO-A-98/58974 and the patent application WO-A-99/31144 from CSIRO relate to the use of dithiocarbamates as “RAFT” reagents. By using these dithiocarbamates, various monomers are polymerized, including vinyl acetate.

The main advantages of the “RAFT” technique over the “ATRP” technique are that “RAFT” does not require a metal catalyst and also that standard radical initiators may be used to initiate the reaction.

In the case of controlled free-radical polymerization, for example of N-vinylpyrrolidone (NVP), chain-transfer agents that may be used in the case of “RAFT” polymerization include, for instance, diphenyldithiocarbamate derivatives.

By varying the ratio of the concentration of monomer to the concentration of chain-transfer agent, the molecular mass of the polymer may be modified.

The polymerization proceeds in several steps according to the general scheme:
a—polymerization of the first monomer or mixture of monomers is performed to form a macroinitiator,
b—the polymers purified by precipitation are dried under vacuum,
c—polymerization of the second block comprising a monomer or a mixture of monomers is performed at the end of the macroinitiator (formed in part a).

Parts b and c are repeated as many times as necessary according to the number of blocks.

One process that may also be used is, for instance, the reverse ATRP polymerization process, which should not be confused with the standard ATRP process described above. In the reverse ATRP process, the initiation is performed in a conventional manner with an initiator capable of giving radicals, for instance azobisisobutyronitrile (AIBN) or a peroxide (rather than using a specific initiator). The presence of metal halides, such as CuBr₂ and of a ligand makes it possible to control the polymerization by reversibly scavenging the radicals formed.

Non-limiting examples of ligands that may be used include amine-based molecules, ifor instance, tris(dimethylaminoethyl)amine (Me₆TREN). Good control of the polymerization is observed for example with the AIBN/CuBr₂/Me₆TREN system.

Another process that may be used, for instance, is the “RAFT” polymerization process.

Second Mode: Free-Radical Polymerization Technique

The block polymers according to the invention may also be obtained by using the standard free-radical polymerization technique, by adding the monomers sequentially. In this case, only control of the nature of the blocks is possible (no control of the masses).

This involves polymerizing, in a first stage, a monomer M1 in a polymerization reactor; kinetically monitoring its consumption over time, and then, when M1 is approximately 95% consumed, introducing a new monomer M2 into the polymerization reactor.

A polymer of block structure of M1-M2 type is thus obtained.

More specifically, a first process for preparing a copolymer as described above comprising at least one block A and at least one block B and optionally at least one other block different from the blocks A and B, in which the polymerization is performed via the reverse atom transfer radical polymerization technique (reverse ATRP) comprises the following:
a) the at least one monomer from which the block A is obtained is polymerized in the presence of a chain-transfer agent, such as a transition metal halide, a free-radical initiator and a ligand, and in the presence or absence of a solvent, by means of which a macroinitiator or functional precursor capable of initiating the polymerization since it comprises the chain-transfer agent functional group at its ends is obtained;
b) the at least one monomer from which the block B is obtained is polymerized at the end of the said macroinitiator or precursor in the presence of a chain-transfer agent such as a transition metal halide, an initiator and a ligand, and in the presence or absence of a solvent, by means of which a diblock copolymer of structure A-b-B is obtained;
c) part b) is optionally repeated with the monomers from which the block or the various other blocks of blocks A and B are prepared, by means of which a triblock or multiblock copolymer is obtained.

It is possible to use difunctional initiators to obtain symmetrical triblock copolymers.

For example, the chain-transfer agents may be chosen from halides of metals in the highest possible oxidation state, for instance, from CuBr₂, CuCl₂, FeCl₂P(phenyl)₃, FeCl₃ and RuCl₂P(phenyl)₃.

Further non-limiting mention may be made of the free-radical polymerization initiators chosen from:
azo compounds, such as 2,2′-azobisisobutyronitrile (AIBN), 2,2′-azobis(2-butanenitrile), 4,4′-azobis(4-pentanoic acid), 1,1′-azobis(cyclohexanecarbonitrile), 2-(t-butylazo)-2-cyanopropane, 2,2′-azobis[2-methyl-N-(1,1)-bis(hydroxymethyl)-2-hydroxyethyl]propionamide, 2,2′-azobis(2-methyl-N-hydroxyethyl)propionamide, 2,2′-azobis(N,N′-dimethyleneisobutyramidine) dichloride, 2,2′-azobis(2-amidinopropane) dichloride, 2,2′-azobis(N,N′-dimethyleneisobutyramide), 2,2′-azobis(2-methyl-N-[1,1-bis(hydroxymethyl)-2-hydroxyethyl]propionamide), 2,2′-azobis(2-methyl-N-[1,1-bis(hydroxymethyl)ethyl]propionamide), 2,2′-azobis(2-methyl-N-(2hydroxyethyl)propionamide), 2,2′-azobis(isobutyramide) dihydrate;
hydrogen peroxides, such as tert-butyl hydroperoxide, cumene hydroperoxide, t-butyl peroxyacetate, t-butyl peroxybenzoate, t-butyl peroxyoctoate, t-butyl peroxyneodecanoate, t-butyl peroxyisobutarate, lauroyl peroxide, t-amyl peroxypivalate, t-butyl peroxypivalate, dicumyl peroxide, benzoyl peroxide;
alkaline persulfates, such as potassium persulfate or ammonium persulfate;
redox systems comprising combinations such as:
   mixtures of hydrogen peroxide, alkyl peroxide, peresters, percarbonates and the like, and any iron salts, titanous salts, zinc formaldehyde sulfoxylate or sodium formaldehyde sulfoxylate, and reducing sugars;
   alkali metal or ammonium persulfates, perborate or perchlorate in combination with an alkali metal bisulfite, such as sodium metabisulfite, and reducing sugars,
   alkali metal persulfates in combination with an arylphosphinic acid, such as benzenephosphonic acid and the like, and reducing sugars.

The ligands may, for example, be chosen from tetradentate ligands, such as 1,1,4,7,10,10-hemamethyltriethylenetetramine (HMTETA), 1,4,8,11-tetraazacyclotetradecane (cyclame), 1,4,8,11-tetramethyl-1,4,8,11-tetraazacyclotetradecane (Me₄ cyclame) and tris(dimethylaminoethyl)amine (Me₆TREN); hexadentate ligands, such as tetrakis(2-pyridyl)pyrazine (TPPY), N,N,N′,N′-tetrakis(2-pyridylmethyl)ethylenediamine (TPMEDA) and triphenylphosphine (TPP).

Non-limiting examples of solvents that may be used include dioxane, tetrahydrofuran (THF), N-methylpyrrolidone, water, and mixtures thereof.

The catalysts, initiators, ligands and solvents may be identical or different in parts a) and b). In one embodiment of the present disclosure for example, they are identical. For instance, in the first and second parts a) and b), the chain-transfer agent may be CuBr₂, the initiator may be AIBN, the ligand may be Me₆TREN and the solvent may be dioxane.

This first process makes it possible to obtain any of the copolymers of the present disclosure, for example, it applies to the preparation of a copolymer in which block A is a copolymer obtained from a majority monomer chosen from N-vinylpyrrolidone and N-vinylcaprolactam, and from a minority monomer that is chosen from t-butyl acrylate and butyl acrylate, for example, and block B is, for example, a homopolymer obtained from a monomer chosen from methyl acrylate, t-butyl acrylate and methyl methacrylate.

A second process for preparing a copolymer as disclosed herein comprising at least one block A and at least one block B and optionally at least one other block different from the blocks A and B, in which the polymerization is performed via the RAFT “Reversible Addition-Fragmentation Chain Transfer” polymerization technique includes the following:
a) the at least one monomer from which block A is obtained is polymerized in the presence of a chain-transfer agent and an initiator, optionally in a solvent, by means of which a macroinitiator or precursor comprising the chain-transfer agent functional group at its ends is obtained;
b) the at least one monomer from which block B is obtained is polymerized at the end of the macroinitiator or precursor, in the presence of an initiator, optionally in a solvent, by means of which a copolymer A-b-B is obtained;
c) part (b) is optionally repeated with the monomers from which the other blocks different from blocks A and B are prepared, by means of which a triblock or multiblock copolymer is obtained.

For example, the chain-transfer agents may be chosen from dithioesters (—C(S)S—) such as dithiobenzoates, dithiocarbamates (—NC(S)S—) or dithiocarbonates (—OC(S)S—) (xanthates). For further example, the chain-transfer agents may be chosen from diethyl malonate diphenyl dithiocarbamate (DPCM), fluoroethyl acetate diphenyldithiocarbamate (DPFEM) and the xanthate of formula C₂H₅OC(S)SCH(CH₃)COOCH₃.

Non-limiting examples of initiators include the compounds already mentioned above for the first process.

Non-limiting examples of solvents include dioxane, tetrahydrofuran, N-methylpyrrolidone, water, and mixtures thereof.

The initiators, chain-transfer agents and solvents may be identical or different in parts a) and b). In one embodiment of the present disclosure, they are identical.

For example, in another embodiment of the present disclosure, in the first and the second parts a) and b), the initiator is AIBN, the chain-transfer agent is chosen from diethyl malonate diphenyldithiocarbamate, (DPCM), fluoroethyl acetate diphenyldithiocarbamate (DPFEM) and the xanthate of formula C₂H₅OC(S)SCH(CH₃)COOCH₃ according to the nature of the monomers to be polymerized, and the solvent is dioxane.

This second process can make it possible to obtain any one of the copolymers of the invention, for example, it can be used to obtain a copolymer in which the at least one block A is a copolymer obtained from a majority monomer chosen from N-vinylpyrrolidone and N-vinylcaprolactam, and from a minority monomer, which is chosen from t-butyl acrylate and butyl acrylate, and the at least one block B is a homopolymer obtained from a monomer chosen from methyl acrylate, t-butyl acrylate and methyl methacrylate.

Another aspect of the present disclosure is cosmetic and pharmaceutical compositions comprising the copolymer as disclosed herein.

Generally, the copolymer as disclosed herein is present in these compositions in an amount ranging from 0.1% to 60% by weight, for instance from 0.5% to 50% by weight, such as from 1% to 40% by weight, relative to the total weight of the composition.

These compositions, for instance cosmetic compositions, according to the present disclosure comprise, besides the copolymer, a physiologically acceptable medium, i.e. a medium that is compatible with keratin materials, for instance the skin, the hair, the eyelashes, the eyebrows and the nails.

In general, it should be considered that the whole composition is physiologically acceptable. The physiologically acceptable medium generally comprises at least one suitable physiologically acceptable solvents, in which the copolymer according to the present disclosure is present in dissolved or dispersed form.

The composition, for example the cosmetic composition, may thus comprise, as suitable solvents forming a hydrophilic phase, at least one solvent chosen from water, and mixtures of water and of at least one hydrophilic organic solvent, for instance alcohols, such as linear or branched lower monoalcohols comprising from 2 to 5 carbon atoms, for instance ethanol, isopropanol or n-propanol, and polyols, for instance glycerol, diglycerol, propylene glycol, sorbitol, pentylene glycol and polyethylene glycols. The hydrophilic phase may also contain C₂ ethers.

The water or the mixture of water and at least one hydrophilic organic solvent may be present in the composition as disclosed herein in an amount ranging from 0% to 90%, for instance, from 0.1% to 90%, by weight, such as from 0% to 60% by weight, for instance from 0.1% to 60% by weight, relative to the total weight of the composition.

The physiologically acceptable medium of the composition may also comprise a fatty phase, comprising, for example, fatty substances that are liquid at room temperature and/or fatty substances that are solid at room temperature, such as waxes, pasty fatty substances and gums, and mixtures thereof. These fatty substances may be of animal, plant, mineral or synthetic origin. The fatty phase may also comprise lipophilic organic solvents.

Among fatty substances that are liquid at room temperature, often known as oils, which may be used according to the present disclosure, non-limiting mention may be made of: hydrocarbon-based oils of animal origin such as perhydrosqualene; hydrocarbon-based plant oils such as liquid triglycerides of fatty acids comprising from 4 to 10 carbon atoms, for instance heptanoic or octanoic acid triglyceride, or alternatively sunflower oil, corn oil, soybean oil, grape seed oil, sesame seed oil, apricot oil, macadamia oil, castor oil, avocado oil, caprylic/capric acid triglycerides, jojoba oil and shea butter oil; linear or branched hydrocarbons of mineral or synthetic origin such as liquid paraffins and derivatives thereof, petroleum jelly, polydecenes and hydrogenated polyisobutene such as parleam; synthetic esters and synthetic ethers, for instance of fatty acids, such as, for example, Purcellin oil, isopropyl myristate, 2-ethylhexyl palmitate, 2-octyldodecyl stearate, 2-octyldodecyl erucate and isostearyl isostearate; hydroxylated esters, for instance isostearyl lactate, octyl hydroxystearate, octyidodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate or diethylene glycol diisononanoate; and pentaerythritol esters; fatty alcohols comprising from 12 to 26 carbon atoms, for instance octyldodecanol, 2-butyloctanol, 2-hexyldecanol, 2-undecylpentadecanol and oleyl alcohol; partially hydrocarbon-based or silicone-based fluoro oils; silicone oils, for instance linear or cyclic, volatile or non-volatile polymethylsiloxanes (PDMSs) that are liquid or pasty at room temperature, for instance cyclomethicones, dimethicones, optionally comprising a phenyl group, for instance phenyl trimethicones, phenyltrimethylsiloxydiphenyl siloxanes, diphenylmethyldimethyltrisiloxanes, diphenyl dimethicones, phenyl dimethicones and polymethylphenylsiloxanes; and mixtures thereof.

The at least one oil, when present, may be present in an amount ranging from 0.01% to 90%, such as from 0.1% to 85% by weight, relative to the total weight of the composition.

The physiologically acceptable medium of the composition according to the present disclosure may also comprise at least one organic solvent that is cosmetically and/or pharmaceutically acceptable, i.e., acceptable tolerability, toxicology and feel. The at least one organic solvent, when present, may be present in an amount ranging from 0% to 90%, for instance, from 0.1% to 90% and from 10% to 90% by weight, such as from 30% to 90%, by weight, relative to the total weight of the composition.

Among the solvents that may be used in the composition of the present disclosure, non-limiting mention may be made of acetic acid esters, for instance methyl acetate, ethyl acetate, butyl acetate, amyl acetate, 2-methoxyethyl acetate or isopropyl acetate; ketones, for instance methyl ethyl ketone or methyl isobutyl ketone; hydrocarbons, for instance toluene, xylene, hexane or heptane; aldehydes comprising from 5 to 10 carbon atoms; ethers comprising at least 3 carbon atoms; and mixtures thereof.

The waxes may be hydrocarbon-based waxes, fluoro waxes and/or silicone waxes and may be of plant, mineral, animal and/or synthetic origin. For example, the waxes can have a melting point of greater than 25° C., such as greater than 45° C.

Among the waxes that may be used in the composition of the present disclosure, non-limiting mention may be made of beeswax, carnauba wax or candelilla wax, paraffin, microcrystalline waxes, ceresin or ozokerite; synthetic waxes, for instance polyethylene waxes or Fischer-Tropsch waxes, or silicone waxes, for instance alkyl dimethicones or alkoxy dimethicones comprising from 16 to 45 carbon atoms.

The gums are generally chosen from polydimethylsiloxanes (PDMSs) of high molecular weight or cellulose gums or polysaccharides, and the pasty substances are generally chosen from hydrocarbon-based compounds, for instance lanolins and derivatives thereof, or PDMSs.

The nature and amount of the solid substances depend on the desired mechanical properties and textures. As a guide, the composition may contain from 0 to 50% by weight, for instance from 1% to 30% by weight of waxes relative to the total weight of the composition.

The copolymer may be combined with at least one auxiliary film-forming agent. Such a film-forming agent may be chosen from any compound known to those skilled in the art as being capable of fulfilling the desired function, and for example, may be chosen from plasticizers and coalescers.

The physiologically acceptable medium of the composition according to the present disclosure may also comprise at least one dyestuff chosen from water-soluble dyes and pulverulent dyestuffs, for instance pigments, nacres and flakes that are known to those skilled in the art. The dyestuffs may be present in the composition in a total amount ranging from 0.01% to 50% by weight, for instance from 0.01% to 30% by weight, relative to the weight of the composition.

As used herein, the term “pigments” should be understood as meaning white or colored, mineral or organic particles of any form, which are insoluble in the physiological medium and which are intended to color the composition.

As used herein, the term “nacres” should be understood as meaning iridescent particles of any form, produced for instance, in the shell of certain molluscs, or alternatively synthesized.

The pigments may be white or colored, and mineral and/or organic. Among the mineral pigments that may be used in the present disclosure, non-limiting mention may be made of are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders, for instance aluminium powder or copper powder.

Among the organic pigments that may be used in the present disclosure, non-limiting mention may be made of carbon black, pigments of D & C type, and lakes based on cochineal carmine or on barium, strontium, calcium or aluminium.

The nacreous pigments may be chosen from white nacreous pigments such as titanium-coated mica or bismuth oxychloride-coated mica, coloured nacreous pigments such as titanium mica coated with iron oxides, titanium mica coated for example, with ferric blue or with chromium oxide, titanium mica coated with an organic pigment of the abovementioned type and also nacreous pigments based on bismuth oxychloride.

The water-soluble dyes are, for example, beetroot juice or methylene blue.

The physiologically acceptable medium of the composition, for example of the cosmetic composition, according to the present disclosure may also comprise at least one filler, for example, in an amount ranging from 0.01% to 50% by weight, such as ranging from 0.01% to 30% by weight, relative to the total weight of the composition. As used herein, the term “fillers” means colorless or white, mineral or synthetic particles of any form, which are insoluble in the medium of the composition, irrespective of the temperature at which the composition is manufactured. These fillers serve for example, to modify the rheology or the texture of the composition.

The fillers may be mineral or organic of any form, platelet, spherical or oblong, irrespective of the crystallographic form (for example lamellar, cubic, hexagonal, orthorhombic, etc.). Non-limiting mention may be made of talc, mica, silica, kaolin, polyamide powder (Nylon®) (Orgasol® from Atochem), poly-β-alanine powder and polyethylene powder, tetrafluoroethylene polymer (Teflon®) powders, lauroyllysine, starch, boron nitride, hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (for example Tospearls® from Toshiba), polyorganosiloxane elastomer particles, precipitated calcium carbonate, magnesium carbonate, magnesium hydrocarbonate, hydroxyapatite, hollow silica microspheres (Silica Beads® from Maprecos), glass or ceramic microcapsules, metal soaps derived from organic carboxylic acids comprising from 8 to 22 carbon atoms, such as from 12 to 18 carbon atoms, for example zinc stearate, magnesium stearate, lithium stearate, zinc laurate or magnesium myristate.

The physiologically acceptable medium of the composition according to the present disclosure may also comprise ingredients commonly used in cosmetics or in pharmaceuticals, such as vitamins, thickeners, trace elements, softeners, sequestering agents, fragrances, acidifying or basifying agents, preserving agents, sunscreens, surfactants, antioxidants, agents for preventing hair loss, antidandruff agents, propellants, and film-forming or non-film-forming water-soluble or liposoluble polymers, or polymers dispersed in water or in a gaseous phase, or mixtures thereof.

Needless to say, a person skilled in the art will take care to select any and all of these optional additional compounds, and/or the amount thereof, such that the beneficial properties of the corresponding composition according to the present disclosure are not, or are not substantially, adversely affected by the envisaged addition.

The composition, for example the cosmetic composition, according to the present disclosure, may be in the form, for example, of a suspension, a dispersion, a solution, a gel, an emulsion, such as an oil-in-water (O/W) or water-in-oil (W/O) emulsion, or a multiple emulsion (W/O/W or polyol/O/W or O/W/O emulsion), in the form of a cream, a paste, a mousse, a dispersion of vesicles, for instance, of ionic or nonionic lipids, a two-phase or multi-phase lotion, a spray, a powder, a paste, such as a soft paste, for instance, a paste having a dynamic viscosity at 25° C. ranging from 0.1 to 40 Pa·s at a shear rate of 200 s⁻¹ after measurement for 10 minutes in cone/plate geometry. The composition may also be anhydrous, for example, it may be an anhydrous paste.

A person skilled in the art can select the appropriate presentation form, and also the method for preparing it, on the basis of his or her general knowledge, taking into account firstly the nature of the constituents used, such as their solubility in the support, and secondly the intended use of the composition.

The composition according to the present disclosure may be a makeup composition, for instance complexion products (foundations), rouges, eye shadows, lip products, concealer products, blushers, mascaras, eyeliners, eyebrow makeup products, lip pencils, eye pencils, nail products, such as nail varnishes, body makeup products or hair makeup products, for instance, hair lacquer or mascara.

The composition according to the present disclosure may also be a hair product, such as for holding the hairstyle or for shaping the hair. The hair compositions can be, for example, shampoos, hairsetting gels or lotions, blow-drying lotions, or fixing and styling compositions such as lacquers or sprays.

When the copolymer according to the present disclosure is used in hair products, such as products for holding the hairstyle or for shaping the hair, it makes it possible to avoid “flaking” powdering.

The products may be packaged in various forms: for example as gels or lotions, and for instance, in vaporizers, in pump-dispenser bottles or in aerosol containers in order to allow the composition to be applied in vaporized form or in the form of a mousse. Such packaging forms are indicated, for example, when it is desired to obtain a spray or a mousse for fixing or treating the hair.

It has been seen hereinabove that the copolymer according to the present disclosure is suitable for use under such conditions since it is highly compatible with the propellant gases and for use, for example, in aerosol containers. The at least one propellant can be chosen from dimethyl ether, C₃₋₅ alkanes, such as propane, n-butane and isobutane; 1,1-difluoroethane, mixtures of dimethyl ether and of C₃₋₅ alkanes, and mixtures of 1,1-difluoroethane and of dimethyl ether and/or of C₃₋₅ alkanes.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The examples below serve to illustrate the present disclosure in a non-limiting manner.

### EXAMPLES

In the examples below, polymers according to the present disclosure are prepared via the reverse atom-transfer radical polymerization technique, also known as reverse ATRP, or via the RAFT (reverse addition-fragmentation chain transfer) polymerization technique.

In general, the formation of blocks in the case of the reverse ATRP process and of the RAFT process take place in several parts.

Thus, in the case of a diblock copolymer, the process will be performed, for example, in the following manner:
a) polymerization of the first monomer to form the macroinitiator or precursor;
b) polymerization of the second monomer at the end of the macroinitiator, to form the diblock.

Between the two parts, a purification step may be necessary, for instance in the case of the reverse ATRP polymerization.

The general procedure used to perform the examples will first be described, for each of the processes: process 1 (reverse ATRP polymerization) and process 2 (RAFT polymerization).

1. Process 1: Reverse ATRP

The reverse ATRP process is performed for example, with tris(dimethylaminoethyl)amine (TREN Me₆) as ligand, using CuBr₂ and employing AIBN as initiator.

1.1 Starting Materials

The ethylenic monomer comprising a lactam ring, such as N-vinylpyrrolidone (VP), obtained from the company Aldrich®, is distilled under vacuum and stored under nitrogen at 0° C. before use.

The other monomers, such as tert-butyl acrylate and methyl methacrylate, obtained from the company Aldrich®, are dried over calcium hydride and distilled under vacuum.

The initiator azobisisobutyronitrile (AIBN) is recrystallized from methanol.

CuBr₂ (99.9%), CuBr and copper powder (99%) are obtained from the company Aldrich® and used as supplied.

The tris(dimethylaminoethyl)amine (TREN Me₆) is synthesized according to the procedures described in the literature, for example in the Matyjaszewski document ACS Symp. Ser. 2000; 760; 207.

All the solvents: THF, dioxane and DMF, are dried by distillation over CaH₂ before use.

1.2. Polymerization

The general procedure is as follows:

1.2.1. Formation of the Macroinitiator or Precursor

CuBr₂ and Me₆ TREN are placed in a round-bottomed flask and dioxane is added thereto.

The solution is stirred for 30 minutes at 25° C.

The at least one monomer (according to the block prepared first and forming the macroinitiator) is added, followed by addition of the initiator AIBN.

The system is subjected to three cycles of vacuum-drying/argon. The solution is then heated in a thermostatically-regulated bath. The viscosity increases. After reaction for the desired time, the system is cooled to room temperature.

The macroinitiator polymer is separated out by precipitation: it is diluted in chloroform and then precipitated from diethyl ether; this operation is repeated twice. The copper complex is separated from the polymer dissolved in the chloroform by passing through a neutral column. The polymer solution is then evaporated.

1.2.2. Polymerization of the Second Block at the End of the Macroinitiator

The macroinitiator obtained in the first step is added to a solution of CuBr and Me₆TREN in dioxane under an inert atmosphere of nitrogen.

The required amount of at least one monomer constituting the second block is added. The solution is heated. After the desired time, the system is cooled to room temperature.

The polymer is separated out by precipitation: it is diluted in chloroform and then precipitated from diethyl ether; this operation is repeated twice. The copper complex is separated from the polymer dissolved in the chloroform by passing through a neutral column. The polymer solution is evaporated.

The polymer is dried.

The isolated polymer is generally a white powder.

2. Process 2: RAFT

Ethylenic monomers comprising a lactam ring, for example vinyllactams, are polymerized with the following compounds as chain-transfer agents:
diethyl malonate diphenyldithiocarbamate (DPCM): 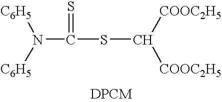
ethyl fluoroacetate diphenyldithiocarbamate (DPFEM) 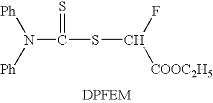
xanthate: C₂H₅ O C(S)S C H(CH₃) COOCH₃
   Starting Materials

The ethylenic monomer comprising a lactam ring, such as N-vinylpyrrolidone (VP) or N-vinylcaprolactam (VCap) is obtained from the company Aldrich® and is distilled under vacuum before use.

The other monomers, such as tert-butyl acrylate and methyl methacrylate, are obtained from the company Aldrich® and are dried over calcium hydride and distilled under vacuum.

The AIBN is obtained from the company Aldrich® and is recrystallized from methanol.

The diphenylamine, the CS₂ and the bromodiethyl malonate are obtained from the company Aldrich® and are used as received.

The solvents such as dioxane are dried by distillation over CaH₂ before use.

2.1. Synthesis of the Chain-Transfer Agent

2.1.1. Synthesis of Diethyl Malonate Diphenyidithiocarbamate (DPCM)

Diethyl malonate diphenyldithiocarbamate (DPCM) is synthesized from diphenylamine and bromodiethyl malonate in the following manner:

1.69 g of diphenylamine (10 mmol) in 10 ml of DMSO and 5 ml of THF are added at 0° C. to a solution of 0.625 g of NaH (purified by washing with dry hexane) in 5 ml of THF (dry). The reaction mixture is stirred for 1.5 hours to give a clear green solution. 1.2 eq of CS₂ (1.42 ml, 1.2 mmol) are added to this solution and the mixture is stirred for 30 minutes at 0° C. to obtain an orange-yellow solution of the sodium salt of diphenyldithiocarbamate.

Bromodiethyl malonate (10 mmol) is added to the above solution at −20° C. and the temperature of the reaction mixture is brought slowly up to room temperature. After stirring for 2 hours at room temperature, the reaction mixture is treated with water and extracted with ether. The ether layer is dried over MgSO₄ and concentrated.

Yield: 51%

The purity of the product was checked by NMR.

2.1.2. Synthesis of Fluoroethyl Acetate Diphenyldithiocarbamate (DPFEA)

Sodium hydride (7 mmol/0.168 g, 1.3 eq in 5 ml of THF) is placed in aflame-dried flask and stirred at 0° C. Diphenylamine (5.4 mmol, 1 eq) in 5 ml of THF and 10 ml of DMSO is added dropwise to this mixture and stirred for 1 hour. Carbon disulfide (2.3 eq) is added to the solution at 0° C., and stirring is continued for a further half an hour. The temperature of the solution is reduced to 18° C. and one equivalent of fluoroethyl acetate is added. After addition, the reaction mixture is warmed slowly to room temperature and stirred for half an hour at room temperature. The product obtained is hydrolyzed by adding water and the organic layer is extracted with ether. The ether extract is concentrated to give yellow crystals of (DPFEA) and the purity of the product is checked by NMR analysis.

2.2. Polymerization

2.2.1. The general procedure for the polymerization is the following, whether the chain-transfer agent is diethyl malonate diphenyldithiocarbamate or fluoroethyl acetate dithiocarbamate. In the text hereinbelow, vinylpyrrolidone is mentioned, but the preparation process can be generalized to any ethylenic monomer comprising a lactam ring.

Preparation of the Precursor (or Macroinitiator): Copoly(Vinylpyrrolidone/Minor Monomer)

The polymerization of vinylpyrrolidone (VP) and of a minority monomer (for example tert-butyl acrylate) (block A) using diethyl malonate diphenyldithiocarbamate as RAFT reagent is performed using AIBN as initiator.

In a typical experiment, VP, the minority monomer of block A (i.e. tert-butyl acrylate), the diethyl malonate diphenyldithiocarbamate (chain-transfer agent/monomer ratio= 1/100), the AIBN (10% of the chain-transfer agent) and dioxane (ratio of 1/1 by volume relative to the monomer) are placed in a Schlenk tube. The reaction mixture is degassed by three freeze-pump-thaw cycles and then sealed hermetically under vacuum and heated in a bath at a constant temperature of 80° C.

The “active” macroinitiator, which may be represented by “X′-copoly(vinylpyrrolidone/minority monomer)-X” wherein X is (phenyl)₂NC(S)S—, and X′ is —CH(COOC₂H₅)₂, obtained is purified by repeated precipitations from diethyl ether and is dried under vacuum.

Polymerization of the Second Block at the End of the “Active” Macroinitiator “X′-copoly(vinylpyrrolidone/minority Monomer)-X”

The polymerization of the second monomer forming the block B (which is, for example, methyl methacrylate) takes place in the presence of the macroinitiator “X′-copoly(vinylpyrrolidone/minor monomer)-X” above (block A), in 1.5 ml of dioxane and in the presence of AIBN (0.1 mol % relative to the total amount of monomer and of macroinitiator).

The reaction mixture is subjected to three freeze-pump-thaw cycles and heated in an oil bath at 80° C. with stirring for 20 hours.

Once the reaction is complete, the reaction mixture is dissolved in dichloromethane (minimum amount required for dissolution) and is precipitated from ether.

The cloudy ether solution is filtered and concentrated by flash evaporation, and is added to pentane; the precipitate obtained is dried under vacuum at about 70° C. for 8 hours.

2.2.2. As a variant, the polymerization may be performed in the following manner: the synthesis of a poly(methyl methacrylate) macroinitiator (block B) is first performed, for example, and the polymerization of block (A) comprising vinylpyrrolidone and of minority monomer such as tert-butyl acrylate is then performed at the end of this macroinitiator. The same procedure as in paragraph 2.2.1. is followed.

2.2.3. Polymerization using a xanthate as chain-transfer agent: Synthesis without an intermediate purification step (“one-pot synthesis”)

N-Vinylpyrrolidone (4 ml, 3.74 eq; 10⁻² mol) (the procedure may also be applied to another ethylenic monomer comprising a lactam ring), the minority monomer: tert-butyl acrylate, the chain-transfer agent (above xanthate, 0.0839 g, chain-transfer agent/monomer ratio is about 1/100 on a molar basis) and dioxane (4 ml) are placed in a predried round-bottomed flask and AIBN (0.0061 g, 10 mol % of the chain-transfer agent) is added thereto under nitrogen.

The reaction mixture is subjected to three freeze-pump-thaw cycles and is heated in an oil bath thermostatically maintained at 80° C., with stirring. The reaction is stopped after 14 hours by cooling in liquid nitrogen.

The second monomer (forming the block B), for example methyl methacrylate (3 ml), and dioxane (3 ml) are added to this reaction mixture at room temperature under nitrogen. The reaction mixture is again subjected to three freeze-pump-thaw cycles and is heated at 80° C. for a further 24 hours.

Once the reaction is complete, the reaction mixture is dissolved in dichloromethane (minimum amount required for dissolution) and is precipitated from ether. The cloudy ether solution is filtered and concentrated by flash evaporation, it is added to pentane and the precipitate obtained is dried under vacuum at about 70° C. for 8 hours.

Characterizations

The conversion is measured by weighing the polymer.

The mass of the macroinitiator based on vinyllactam, for example the copoly(vinylpyrrolidone/tert-butyl acrylate), is determined by gas chromatography GC (Varian 9012® machine) with G4000 G3000 G2500 TSK® gel columns, equipped with a Varian® RI 4 infrared detector. The eluent is an 80/20 water/methanol mixture containing 0.1 M sodium nitrate. The flow rate of the eluent is 0.5 ml/minute. The calibration is performed with poly(ethylene oxide) standards.

The mass measurement is therefore only a comparative measurement and the analogous values do not represent the actual molecular masses. However, the results may be used to note the trend in the change of the molecular masses and also to determine the mass dispersity of the chains.

The mass of the PMMA macroinitiator is determined by GC (Varian® 9012) with G4000 G3000 G2500 TSK® gel columns, equipped with a Varian® RI 4 infrared detector. The eluent is THF. The standard is polystyrene.

The overall mass of the copolymer is determined by GC chromatography in solvent phase (Varian®) 9012) with G4000 G3000 G2500 TSK® gel columns, equipped with a Varian® RI 4 infrared detector. The eluent is THF. The standard is polystyrene.

The proportion of the various blocks is determined by ¹H NMR (Bruker®, 200 MHz), by determining the ratio of the areas of the peaks corresponding to the monomers of the various blocks (i.e., on the one hand MMA and on the other hand VP, for example).

After having described above the general procedure for the synthesis of the copolymers according to the present disclosures, examples concerning the preparation of a specific copolymer in accordance with the invention (Example 3) and also two comparative examples are given below.

Comparative Example 1

In this example, a copolymer (not in accordance with the present disclosure) comprising a polyvinylpyrrolidone block and a poly(methyl methacrylate) block: (PVP) 90%-b-(PMMA) 10% was prepared according to process 1 (reverse ATRP) described above.

Synthesis of the Polyvinylpyrrolidone-Br Macroinitiator: PVP-Br

CuBr₂ (0.0314 g, 1.4×10⁻⁴ mol) and Me₆TREN (0.0970 g, 4.2×10⁻⁴ mol) were placed in a 50 ml round-bottomed flask and 5 ml of dioxane were added. N-Vinylpyrrolidone (NVP) (5.2 g, 0.0468 mol) was then added, followed by addition of AIBN (azobisisobutyronitrile) (0.0154 g, 9.36×10⁻⁵ mol).

The dioxane/monomer ratio was 1/1 by volume.

The AIBN/CuBr₂/CuO/Me₆TREN proportions were: 1/1.5/0.15/3. The reaction was performed at a temperature of 80° C. for 3 hours 45 minutes. The conversion reaches 97%.

Synthesis of the Copolymer: Polymerization of Methyl Methacrylate (MMA) at the End of the Polyvinylpyrrolidone Macroinitiator (PVP-Br)

The PVP macroinitiator (PVP-Br) was added to CuBr (PVP-Br/CuBr ratio=1/1.5) and Me₆TREN (1/3 CuBr/Me₆TREN) in dioxane under a nitrogen atmosphere. Polyvinylpyrrolidone macroinitiator PVP-Br: 0.041 mM/0.92 g. Methyl methacrylate (MMA): 4.09 mM: 0.4 g. Solvent: dioxane: 1.5 ml.

The reaction was performed at a temperature of 100° C. for 14 hours.

The conversion was 51%.

The characteristics of the macroinitiator and of the final copolymer are summarized in the following table:

| | | | % PVP/ | % PMMA/ | Mn |
|---|---|---|---|---|---|
| | | Mn (PVP | total | total | PMMA |
| Co- | VP/ | macroinitiator) | weight | weight | deduced |
| polymer | AIBN | (GC) | NMR | NMR | by NMR |
| (PVP) | 1/0.35 | 24 400 | 87.5% | 12.5% | 9600 |
| 90%-b- | | | | | |
| (PMMA) | | | | | |
| 10% | | | | | |

Vinylpyrrolidone = VP
Azobisisobutyronitrile = AIBN
Mₙ = number-average molecular mass

### Comparative Example 2

In this example, a copolymer (not in accordance with the present disclosure) comprising a polyvinylpyrrolidone (PVP) block and a polystyrene (PS) polymer block was prepared: (PVP) 80%-b-(PS) 20%.

The desired polymer was prepared via a process similar to the one described in paragraph 2.2.2. above, the monomers being adapted to obtain the desired polymer.

Synthesis of the Polystyrene Macroinitiator

Chain-transfer agent: DPCM

Monomer/chain-transfer agent ratio: 200 (on a molar basis)

Solvent: dioxane

Reaction temperature: 100° C.

Polymerization of the Second Block at the End of the Polystyrene Macroinitiator

The reaction mixture comprised 0.5 g of the polystyrene macroinitiator prepared above and 0.25 g of vinylpyrrolidone in 1.5 ml of dioxane, and the reaction was performed in the presence of AIBN (0.1 mol % relative to the styrene).

The conversion was 50%.

The characteristics of the final copolymer are summarized in the following table:

| | % | | % PS/ | Mn of | Mn |
|---|---|---|---|---|---|
| Mn | PVP/total | % PS/total | total | PVP | Mw |
| polystyrene | weight | moles | weight | deduced | Mp |
| (NMR) | NMR | NMR | NMR | by (NMR) | (GC) |
| 4150 | 79 | 29 | 21 | 15330 | Mw 40 500 |
| | | | | | Mn 22 700 |
| | | | | | Mp 41 300 |
| | | | | | Ip 1.8 |

Mn = number-average molecular mass

### Inventive Example 3 (Copolymer of the Present Disclosure)

In this example, a copolymer (in accordance with the present disclosure) comprising a block A (80% by weight) comprising a copolymer based on 70% vinylpyrrolidone (PVP) and 30% t-butyl acrylate, and a block B (20% by weight) comprising poly(methyl methacrylate) was prepared.

The desired polymer was prepared via a process similar to the one described in paragraph 2.2.3. above. The monomer mixture is suitable for obtaining the desired polymer.

The N-vinylpyrrolidone (VP) and the tert-butyl acrylate (3.74×10⁻² mol in total), the chain-transfer agent (xanthate described above, 0.0839 g, chain-transfer agent/monomer ratio: about 1/100) and dioxane (4 ml) were placed in a predried flask, and AIBN (0.0061 g, 10 mol % relative to the chain-transfer agent) was added to the flask under nitrogen.

This reaction mixture was subjected to three freeze-pump-thaw cycles and was heated in an oil bath thermostatically maintained at 80° C., with stirring.

The reaction was stopped after 14 hours by cooling in liquid nitrogen.

The monomer of the second block (methyl methacrylate) (3 ml) and dioxane (3 ml) were added to this reaction mixture at room temperature, under nitrogen. The reaction mixture was again subjected to three freeze-pump-thaw cycles and was heated at 80° C. for a further 24 hours.

Once the reaction was complete, the reaction mixture was dissolved in dichloromethane (minimum amount required for dissolution) and was precipitated from ether.

The cloudy ether solution was filtered and concentrated by flash evaporation, and was added to pentane; the precipitate obtained was dried under vacuum at about 70° C. for 8 hours, and was characterized by GC and NMR.

The characteristics of the reagents and of the process are summarized below.
Chain-transfer agent: xanthate C₂H₅OC(S)SCH(CH₃)COOCH₃.
Molar ratio: xanthate/monomers: 1/100.
AIBN/xanthate chain-transfer agent ratio: 10 mol %.
Monomers/AIBN ratio: 0.1
Reaction temperature: 80° C.
Solvent: dioxane.
Vinylpyrrolidone (VP): 0.027 mol, i.e. 3 g.
tert-Butyl acrylate (tBuA): 0.0098 mol, i.e. 1.254 g.

The characteristics of the copolymer are summarized in the following table:

| | | Mn [poly | | | | |
|---|---|---|---|---|---|---|
| | | [poly(VP- | | % | | % [poly |
| | VP/ | co-tBuA)] | Conversion | PMMA/ | Mn PMMA | (VP-co- |
| | tBuA by | measured | of | total | deduced by | tBuA)]/total |
| Copolymer | weight | by GC | MMA | weight | NMR | weight |
| Poly(VP: 70%: | 70/30 | 6500 | 90% | 25 | 2160 | 75 |
| t-butyl acrylate: | | | | | | |
| 30%) 80% | | | | | | |
| b-(polymeth-yl | | | | | | |
| methacrylate) | | | | | | |
| 20% | | | | | | |

Mₙ = number-average molecular mass

### Example 4

In this example, the tack of the copolymers prepared in Comparative Examples 1 and 2 and in Example 3 was measured.

A polymer film was prepared from a solution in ethanol, on a glass support. The polymer was dissolved at a concentration of 10% in the solvent.

The film is dried in a chamber under controlled humidity.

The tack is evaluated by contact with the finger, on a scale from 0 to 10, wherein 0=no tack, 5=moderate tack, and 10=high tack.

### Example 3 (Inventive)

Value of 2 at ambient RH,

Value of 4 at RH: 100

### Comparative Example 1

Value of 5 at ambient RH

Value of 10 at RH: 100

### Comparative Example 2

Value of 4 at ambient RH

Value of 8 at RH: 100

The polymer of Example 3 in accordance with the present disclosure had a lower tack than the polymers of the comparative examples not in accordance with the present disclosure, both under ambient humidity conditions and under high humidity conditions (RH: 100).

### Example 5

In this example, a spray was formulated with the polymer in accordance with the present disclosure of Example 3.

The polymer of Example 3 was dissolved at a concentration of 6% in ethanol.

The polymer was applied as a spray to the head of hair.

The hairstyle showed no tack, even at high humidity (RH>50%).

1. A linear block ethylenic copolymer comprising:
at least one block A that is obtained from at least one monomer comprising
a majority amount of an ethylenic monomer comprising a lactam ring chosen from monomers of formula (I): 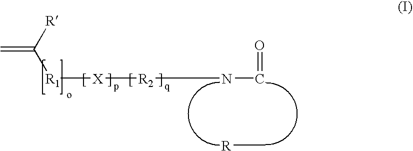
wherein:
R is chosen from —(CH₂)ₙ— radicals, in which at least one carbon atom is optionally replaced with a heteroatom chosen from nitrogen and oxygen atoms and is optionally substituted with at least one C₁ to C₆ alkyl group and wherein n is an integer ranging from 3 to 12;
R′ is chosen from a hydrogen atom and methyl groups;
R₁ and R₂, which may be identical or different, are chosen from linear, branched and cyclic alkylene and aralkylene groups of 1 to 22 carbon atoms, wherein at least one carbon atom is optionally replaced with a heteroatom chosen from oxygen and nitrogen atoms;
X is chosen from —OCO—, —NHCO—, and —COO— groups and an oxygen atom;
o, p and q represent, independently of each other, 0 or 1; and
a minority amount of at least one non-hydrophilic monomer; and
and at least one block B that is obtained from monomers not comprising an ethylenic monomer comprising a lactam ring of formula (I), or comprising a minority proportion thereof.

2. The linear block ethylenic copolymer according to claim 1, wherein, in formula (I), o=0, p=1, q=1, R₂ is —CH₂CH₂—, X is chosen from COO and CONH groups and R is chosen from (CH₂)₃, (CH₂)₅, and (CH₂CH₂NH) groups.

3. The linear block ethylenic copolymer according to claim 1, wherein the at least one ethylenic monomer comprising a lactam ring is chosen from vinyllactams of formula (II): 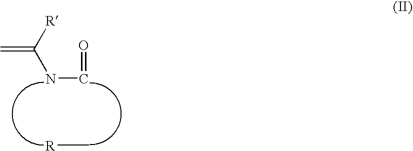
wherein
R is chosen from —(CH₂)ₙ— radicals, in which at least one carbon atom is optionally replaced with a heteroatom chosen from nitrogen and oxygen atoms and is optionally substituted with at least one C₁ to C₆ alkyl group, and wherein n is an integer ranging from 3 to 12; and
R′ is chosen from a hydrogen atom and methyl groups.

4. The linear block ethylenic copolymer according to claim 1, wherein R is —(CH₂)ₙ—, and wherein n is an integer ranging from 3 to 5.

5. The linear block ethylenic copolymer according to claim 4, wherein R is —CH₂—CH₂—NH—.

6. The linear block ethylenic copolymer according to claim 1, wherein the linear block ethylenic copolymer is a film-forming copolymer.

7. The linear block ethylenic copolymer according to claim 1, wherein the number-average molecular mass of the linear block ethylenic copolymer ranges from 4,000 to 1,000,000.

8. The linear block ethylenic copolymer according to claim 7, wherein the number-average molecular mass of the linear block ethylenic copolymer ranges from 4,000 to 800,000.

9. The linear block ethylenic copolymer according to claim 8, wherein the number-average molecular mass of the linear block ethylenic copolymer ranges from 4,000 to 500,000.

10. The linear block ethylenic copolymer according to claim 1, wherein the at least one block A, comprising a majority amount of ethylenic monomer comprising a lactam ring, is present in the copolymer in an amount ranging from 1% to 99% by weight, relative to the total weight of the copolymer.

11. The linear block ethylenic copolymer according to claim 10, wherein the at least one block A, comprising a majority amount of ethylenic monomer comprising a lactam ring, is present in the copolymer in an amount ranging from 10% to 95% by weight, relative to the total weight of the copolymer.

12. The linear block ethylenic copolymer according to claim 11, wherein the at least one block A, comprising a majority amount of ethylenic monomer comprising a lactam ring, is present in the copolymer in an amount ranging from 20% to 90% by weight, relative to the total weight of the copolymer.

13. The linear block ethylenic copolymer according to claim 1, wherein the at least one block B is present in the copolymer in an amount ranging from 1% to 99% by weight, relative to the total weight of the copolymer.

14. The linear block ethylenic copolymer according to claim 13, wherein the at least one block B is present in the copolymer in an amount ranging from 5% to 90% by weight, relative to the total weight of the copolymer.

15. The linear block ethylenic copolymer according to claim 14, wherein the at least one block B is present in the copolymer in an amount ranging from 10% to 80% by weight, relative to the total weight of the copolymer.

16. The linear block ethylenic copolymer according to claim 1, wherein the number-average molecular mass of each block A or B ranges from 2,000 to 1,000,000.

17. The linear block ethylenic copolymer according to claim 16, wherein the number-average molecular mass of each block A or B ranges from 2,000 to 800,000.

18. The linear block ethylenic copolymer according to claim 17, wherein the number-average molecular mass of each block A or B ranges from 2,000 to 500 000.

19. The linear block ethylenic copolymer according to claim 1 wherein the monomers from which the at least one block A is obtained comprise an amount of ethylenic monomer comprising a lactam ring of formula (I), ranging from 50% to 99% by weight, relative to the total amount of monomers of the at least one block A.

20. The linear block ethylenic copolymer according to claim 19, wherein the monomers from which the at least one block A is obtained comprise an amount of ethylenic monomer comprising a lactam ring of formula (I), ranging from 52% to 99%, by weight, relative to the total amount of monomers of the at least one block A.

21. The linear block ethylenic copolymer according to claim 20, wherein the monomers from which the at least one block A is obtained comprise an amount of ethylenic monomer comprising a lactam ring of formula (I), ranging from 55% to 95%, by weight, relative to the total amount of monomers of the at least one block A.

22. The linear block ethylenic copolymer according to claim 21, wherein the monomers from which the at least one block A is obtained comprise an amount of ethylenic monomer comprising a lactam ring of formula (I), ranging from 60% to 80%, by weight, relative to the total amount of monomers of the at least one block A.

23. The linear block ethylenic copolymer according to claim 22, wherein the monomers from which the at least one block A is obtained comprise an amount of ethylenic monomer comprising a lactam ring of formula (I), ranging from 65% to 75%, by weight, relative to the total amount of monomers of the at least one block A.

24. The linear block ethylenic copolymer according to claim 23, wherein the monomers from which the at least one block A is obtained comprise an ethylenic monomer comprising a lactam ring of formula (I), in an amount of 70%, by weight, relative to the total amount of monomers of the at least one block A.

25. The linear block ethylenic copolymer according to claim 1, wherein the at least one block A has an overall glass transition temperature ranging from 0 C to 250° C.

26. The linear block ethylenic copolymer according to claim 25, wherein the at least one block A has an overall glass transition temperature ranging from 0 C to 220° C.

27. The linear block ethylenic copolymer according to claim 26, wherein the at least one block A has an overall glass transition temperature ranging from 5 C to 200° C.

28. The linear block ethylenic copolymer according to claim 1, wherein the N-vinyllactam of formula (I) is chosen from pyrrilidinoethyl acrylate and pyrrilidinoethyl methacrylate.

29. The linear block ethylenic copolymer according to claims 3, wherein the N-vinyllactam of formula (II) is chosen from N-vinylpyrrolidone, N-vinylpiperidinone (valerolactam), N-vinylcaprolactam, N-vinylimidazolidinone wherein R is a —CH₂—CH₂-NH-group, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam and N-vinyl-7-ethyl-2-caprolactam.

30. The linear block ethylenic copolymer according to claim 1, wherein the at least one block A is chosen from random, alternating and gradient copolymers.

31. The linear block ethylenic copolymer according to claim 1, wherein the at least one non-hydrophilic minority monomer of the at least one block A is chosen from:
ethylenic hydrocarbons of 2 to 10 carbon atoms,;
the acrylates of formula CH₂═CHCOOR₃;
the methacrylates of formula: 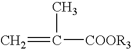
wherein R₃ is chosen from:
linear and branched alkyl groups of 1 to 18 carbon atoms in which at least one heteroatom chosen from O, N, S and P is optionally intercalated, wherein the said alkyl group is optionally substituted with at least one substituent chosen from hydroxyl groups, halogen atoms, and Si(R₄R₅) groups, wherein R₄ and R₅, which may be identical or different, are chosen from C₁ to C₆ alkyl groups and phenyl groups,
C₃ to C₁₂ cycloalkyl groups,
C₃ to C₂₀ aryl groups,
C₄ to C₃₀ aralkyl groups, wherein the alkyl group ranges from C, to C₈,
4- to 12-membered heterocyclic groups comprising at least one heteroatom chosen from O, N and S, wherein the ring is aromatic or non-aromatic,
heterocyclylalkyl groups, wherein the alkyl group ranges from 1 to 4 carbon atoms, and
wherein the cycloalkyl, aryl, aralkyl, heterocyclic and heterocyclylalkyl groups are optionally substituted with at least one substituent chosen from hydroxyl groups, halogen atoms and linear and branched alkyl groups of 1 to 4 carbon atoms in which at least one heteroatom chosen from O, N, S and P is optionally intercalated, wherein the alkyl groups are optionally substituted with at least one substituent chosen from hydroxyl groups, halogen atoms, and Si(R₄R₅) groups, in which R₄ and R₅, which may be identical or different, are chosen from C₁ to C₆ alkyl groups and phenyl groups,
the (meth)acrylamides of formula: 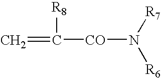
wherein R₈ is chosen from a hydrogen atom and methyl groups, and R₇ and R₆, which may be identical or different, are chosen from
hydrogen atoms and linear and branched alkyl groups of 1 to 18 carbon atoms, in which at least one heteroatom chosen from O, N, S and P is optionally intercalated, and wherein the alkyl group is optionally substituted with at least one substituent chosen from hydroxyl groups, halogen atoms, and Si(R₄R₅) groups, in which R₄ and R₅, which may be identical or different, are chosen from C₁ to C₆ alkyl groups and phenyl groups,
a C₃ to C₁₂ cycloalkyl group,
a C₃ to C₂₀ aryl group,
a C₄ to C₃₀ aralkyl group (C₁ to C₈ alkyl group),
a 4- to 12-membered heterocyclic group containing one or more hetero atoms chosen from O, N and S, the ring being aromatic or non-aromatic,
a heterocyclylalkyl group (1 to 4 C alkyl),
the said cycloalkyl, aryl, aralkyl, heterocyclic or heterocyclylalkyl groups possibly being optionally substituted with at least one substituent chosen from hydroxyl groups, halogen atoms and linear or branched alkyl groups of 1 to 4 carbons, in which at least one hetero atom chosen from O, N, S and P is optionally intercalated, the alkyl groups also possibly being optionally substituted with at least one substituent chosen from hydroxyl groups, halogen atoms and groups Si(R₄R₅), in which R₄ and R₅, which may be identical or different, represent a are chosen from C₁ to C₆ alkyl group and phenyl groups,
the allylic compounds of formula:
CH₂═CH—CH₂—R₉ or CH₂═C(CH₃)—CH₂—R₉;
the vinyl compounds of formula:
CH₂═CH—R₉,
wherein R₉ is a group chosen from:
OR₁₀, wherein R₁₀ is chosen from phenyl groups and C₁ to C₁₂ alkyl groups,
OCOR₁₁, wherein R₁₁ is chosen from:
linear and branched alkyl groups of 2 to 12 carbon atoms,
C₃ to C₁₂ cycloalkyl groups,
C₃ to C₂₀ aryl groups,
C₄ to C₃₀ aralkyl groups, wherein the alkyl group ranges from C₁ to C₈,
4- to 12-membered heterocyclic groups comprising at least one heteroatom chosen from O, N and S, the ring being aromatic or non-aromatic,
heterocyclylalkyl group, wherein the alkyl group ranges from 1 to 4 carbon atoms, and
wherein the cycloalkyl, aryl, aralkyl, heterocyclic and heterocyclylalkyl groups are optionally substituted with at least one substituent chosen from hydroxyl groups, halogen atoms and linear and branched alkyl groups of 1 to 4 carbon atoms in which at least one heteroatom chosen from O, N, S and P is optionally intercalated, wherein the alkyl groups are optionally substituted with at least one substituent chosen from hydroxyl groups, halogen atoms, and Si(R₄R₅) groups, in which R₄ and R₅, which may be identical or different, are chosen from C₁ to C₆ alkyl groups and phenyl groups;
(meth)acrylic or (meth)acrylamide or vinyl monomers comprising a fluoro or perfluoro group;
silicone (meth)acrylic or vinyl monomers.

32. The linear block ethylenic copolymer according to claim 1, wherein the at least one minority monomer, other than the ethylenic monomer comprising a lactam ring, from which the at least one block A is obtained, is chosen from monomers for which the glass transition temperature Tg of the corresponding homopolymer is low.

33. The linear block ethylenic copolymer according to claim 32 wherein the at least one minority monomer, other than the ethylenic monomer comprising a lactam ring, from which the at least one block A is obtained, is chosen from monomers for which the glass transition temperature Tg of the corresponding homopolymer is less than or equal to 50° C.

34. The linear block ethylenic copolymer according to claim 33, wherein the at least one minority monomer, other than the ethylenic monomer comprising a lactam ring, from which the at least one block A is obtained, is chosen from monomers for which the glass transition temperature Tg of the corresponding homopolymer is less than or equal to 20° C.

35. The linear block ethylenic copolymer according to claim 34, wherein the at least one minority monomer, other than the ethylenic monomer comprising a lactam ring, from which the at least one block A is obtained, is chosen from monomers for which the glass transition temperature Tg of the corresponding homopolymer is less than or equal to 0° C.

36. The linear block ethylenic copolymer according to claim 33, wherein the at least one minority monomer, other than the ethylenic monomer comprising a lactam ring, also has a Tg for the corresponding homopolymer of greater than or equal to −150° C.

37. The linear block ethylenic copolymer according to claim 32, wherein the at least one minority monomer, other than the ethylenic monomer comprising a lactam ring, from which the at least one block A is obtained, is chosen from the following monomers for which the glass transition temperature Tg of the corresponding homopolymer is less than or equal to 50° C.: methyl acrylate, ethyl acrylate, n-butyl acrylate, t-butyl acrylate, ethylhexyl acrylate, isobutyl acrylate, butyl methacrylate, n-hexyl methacrylate, POE methacrylate, and vinyl acetate.

38. The linear block ethylenic copolymer according to claim 1, wherein the at least one minority monomer, other than the ethylenic monomer comprising a lactam ring from which the at least one block A is obtained, is chosen from the following monomers: methyl methacrylate, ethyl methacrylate, isobutyl methacrylate, furfuryl acrylate, isobornyl acrylate, tert-butylcyclohexyl acrylate, styrene and vinylcyclohexane.

39. The linear block ethylenic copolymer according to claim 1, wherein the at least one block B other than the at least one block A may be obtained from at least one ethylenic monomer chosen from: allylic monomers, acrylates, methacrylates, acrylamides, methacrylamides, vinyl monomers, ethylenic monomers comprising a lactam ring of formula (I), and mixtures thereof, with the proviso that ethylenic monomers comprising a lactam ring of formula (I), if present in the at least one block B, are present in an amount less than 50% by weight.

40. The linear block ethylenic copolymer according to claim 39, wherein if the ethylenic monomers comprising a lactam ring of formula (I) are present in the at least one block B, they are present in an amount less than or equal to 45% by weight.

41. The linear block ethylenic copolymer according to claim 40, wherein if the ethylenic monomers comprising a lactam ring of formula (I) are present in the at least one block B, they are present in an amount less than or equal to 40% by weight.

42. The linear block ethylenic copolymer according to claim 41, wherein if the ethylenic monomers comprising a lactam ring of formula (I) are present in the at least one block B, they are present in an amount less than or equal to 30% by weight.

43. The linear block ethylenic copolymer according claim 1, wherein the at least one monomer from which the at least one block B is obtained is chosen from non-hydrophilic monomers and hydrophilic monomers.

44. The linear block ethylenic copolymer according to claim 1, wherein the at least one block B has a glass transition temperature of less than or equal to 50° C.

45. The linear block ethylenic copolymer according to claim 44, wherein the at least one block B has a glass transition temperature of less than or equal to 20° C.

46. The linear block ethylenic copolymer according to claim 45, wherein the atl least one block B has a glass transition temperature of less than or equal to 0° C.

47. The linear block ethylenic copolymer according to claim 1, wherein the at least one monomer from which the at least one block B is obtained is chosen from monomers for which the glass transition temperature Tg of the corresponding homopolymer is low.

48. The linear block ethylenic copolymer according to claim 47, wherein the at least one monomer from which the at least one block B is obtained is chosen from monomers for which the glass transition temperature Tg of the corresponding homopolymer is less than or equal to 50° C.

49. The linear block ethylenic copolymer according to claim 48, wherein the at least one monomer from which the at least one block B is obtained is chosen from monomers for which the glass transition temperature Tg of the corresponding homopolymer is less than or equal to 20° C.

50. The linear block ethylenic copolymer according to claim 49, wherein the at least one monomer from which the at least one block B is obtained is chosen from monomers for which the glass transition temperature Tg of the corresponding homopolymer is less than or equal to 0° C.

51. The linear block ethylenic copolymer according to claim 1, wherein the at least one monomer from which the at least one block B is obtained has a Tg for the corresponding homopolymer of greater than −150° C.

52. The linear block ethylenic copolymer according to claim 43, wherein the at least one hydrophilic monomer is chosen from ionic monomers; nonionic monomers; and monomers that may be made hydrophilic as a result of hydrolysis.

53. The linear block ethylenic copolymer according to claim 52, wherein the ionic monomers are chosen from cationic monomers, anionic monomers and betaines.

54. The linear block ethylenic copolymer according to claim 43, wherein the non-hydrophilic monomers from which the at least one block B is obtained are chosen from the monomers for which the glass transition temperature Tg of the corresponding homopolymer is less than or equal to 50° C.: methyl acrylate, ethyl acrylate, n-butyl acrylate t-butyl acrylate, ethylhexyl acrylate, isobutyl acrylate, butyl methacrylate, n-hexyl methacrylate, POE methacrylate, and vinyl acetate.

55. The linear block ethylenic copolymer according to claim 43, wherein the non-hydrophilic monomers from which the at least one block B is obtained, other than the ethylenic monomer comprising a lactam ring from which the block A is obtained, are chosen from the following monomers: methyl methacrylate, ethyl methacrylate, isobutyl methacrylate, furfuryl acrylate, isobornyl acrylate, tert-butylcyclohexyl acrylate, styrene and vinylcyclohexane.

56. The linear block ethylenic copolymer according to claim 53, wherein the cationic monomers are chosen from 2-vinylpyridine; 4-vinylpyridine; dimethylaminoethyl (meth)acrylate; diethylaminoethyl (meth)acrylate; dimethylaminopropyl (meth)acrylamide; and salified or quaternized forms thereof.

57. The linear block ethylenic copolymer according to claim 53, wherein the anionic monomers are chosen from acrylic acid, methacrylic acid, crotonic acid, maleic anhydride, itaconic acid, fumaric acid and maleic acid; styrenesulfonic acid, 2-acrylamido-2-methylpropanesulfonic acid, vinylsulfonic acid, vinylbenzoic acid, sulfopropyl methacrylate and vinylphosphonic acid, and the salts thereof.

58. The linear block ethylenic copolymer according to claim 53, wherein the ionic monomers of betaine type are chosen from ethylenic carboxybetaines and sulfobetaines obtained by quaternization of ethylenically unsaturated monomers comprising an amine function with carboxylic acid salts containing a labile halogen or with cyclic sulfones.

59. (canceled)

60. The linear block ethylenic copolymer according to claim 52, wherein the nonionic monomers are chosen from:
hydroxyalkyl (meth)acrylates and hydroxyalkyl(meth)acrylamides, the alkyl group of which comprises from 2 to 4 carbon atoms;
(meth)acrylates and (meth)acrylamides of (C₁-C₄)alkoxy(C₁₋₄)alkyl;
(meth)acrylates or (meth)acrylamides comprising a group —(OC₂H₄)ₘ—OR′″, in which m is an integer ranging from 5 to 150 and R′″ is chosen from a hydrogen atom and C₁ to C₄ alkyl groups;
vinyllactams, and
polysaccharide (meth)acrylates.

61. The linear block ethylenic copolymer according to claim 1, chosen from:
diblock copolymers (AB);
triblock copolymers chosen from ABA, BAB, ABC and ACB, wherein C is other than A or B,
multiblock copolymers comprising more than three blocks chosen from (AB)ₙ, (ABA)ₙ, (BAB)ₙ, (ABC)N and (ACB)ₙ, wherein C is other than A or B, and multiblock copolymers comprising more than three different blocks, of ABCD type.

62. A cosmetic or pharmaceutical composition comprising a linear block ethylenic copolymer comprising:
at least one block A that is obtained from at least one monomer comprising
a majority amount of an ethylenic monomer comprising a lactam ring chosen from monomers of formula (I): 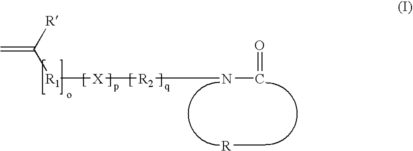
wherein:
R is chosen from —(CH₂)ₙ— radicals, in which at least one carbon atom is optionally replaced with a heteroatom chosen from nitrogen and oxygen atoms and is optionally substituted with at least one C₁ to C₆ alkyl group, and wherein n is an integer ranging from 3 to 12;
R′ is chosen from a hydrogen atom and methyl groups;
R₁ and R₂, which may be identical or different, are chosen from linear, branched and cyclic alkylene and aralkylene groups of 1 to 22 carbon atoms, wherein at least one carbon atom is optionally replaced with a heteroatom chosen from oxygen and nitrogen atoms;
X is chosen from —OCO—, —NHCO—, and —COO— groups and an oxygen atom;
o, p and q represent, independently of each other, 0 or 1; and
a minority amount of at least one non-hydrophilic monomer;
and at least one block B that is obtained from monomers not comprising an ethylenic monomer comprising a lactam ring of formula (I), or comprising a minority proportion thereof.

63. The cosmetic or pharmaceutical composition according to claim 61, wherein the linear ethylenic copolymer is present in an amount ranging from 0.1% to 60% by weight, relative to the total weight of the composition.

64. The cosmetic or pharmaceutical composition according to claim 63, wherein the linear ethylenic copolymer is present in an amount ranging from 0.5% to 50% by weight, relative to the total weight of the composition

65. The cosmetic or pharmaceutical composition according to claim 64, wherein the linear ethylenic copolymer is present in an amount ranging from 1% to 40% by weight, relative to the total weight of the composition

66. The cosmetic or pharmaceutical composition according to claim 62, further comprising a physiologically acceptable medium in which the linear ethylenic copolymer is in dissolved or dispersed form.

67. The cosmetic or pharmaceutical composition according to claim 66, wherein the physiologically acceptable medium comprises at least one suitable solvent forming a hydrophilic phase, chosen from water and mixtures of water and of at least one hydrophilic organic solvent.

68. The cosmetic or pharmaceutical composition according to claim 67, wherein the least one hydrophilic organic solvent is chosen from alcohols and polyols.

69. The cosmetic or pharmaceutical composition according to claim 68, wherein the alcohols are chosen from linear and branched lower monoalcohols comprising from 2 to 5 carbon atoms.

70. The cosmetic or pharmaceutical composition according to claim 69, wherein the polyols are chosen from glycerol, diglycerol, propylene glycol, sorbitol, pentylene glycol and polyethylene glycols.

71. The cosmetic or pharmaceutical composition according to claim 67, wherein the hydrophilic phase further comprises carbon ethers.

72. The cosmetic or pharmaceutical composition according to claim 66, wherein the physiologically acceptable medium further comprises a fatty phase comprising fatty substances that are liquid at room temperature, and/or fatty substances that are solid at room temperature, of animal, plant, mineral or synthetic origin.

73. The cosmetic or pharmaceutical composition according to any one of claim 66, wherein the physiologically acceptable medium comprises at least one cosmetically and/or pharmaceutically acceptable organic solvent.

74. The cosmetic or pharmaceutical composition according to claim 66, wherein the physiologically acceptable medium comprises at least one auxiliary film-forming agent chosen from plasticizers and coalescers.

75. The cosmetic or pharmaceutical composition according to claim 66, wherein the physiologically acceptable medium comprises at least one dyestuff chosen from water-soluble dyes and pulverulent dyestuffs.

76. The cosmetic or pharmaceutical composition according to claim 66, wherein the physiologically acceptable medium comprises at least one filler.

77. The cosmetic or pharmaceutical composition according to claim 66, wherein the physiologically acceptable medium comprises at least one ingredients commonly used in cosmetics and/or pharmaceuticals chosen from vitamins, thickeners, trace elements, softeners, sequestering agents, fragrances, acidifying or basifying agents, preserving agents, sunscreens, surfactants, antioxidants, agents for preventing hair loss, antidandruff agents, propellants, and fibrogeneous or non-fibrogeneous water-soluble or liposoluble polymers or polymers dispersed in water or in an aqueous phase, and mixtures thereof.

78. The cosmetic or pharmaceutical composition according to claim 62, wherein the composition is in a form chosen from a suspension, a dispersion, a solution, a gel, an emulsion, a cream, a paste, a mousse, a dispersion of vesicles, a two-phase or multi-phase lotion, a spray, a powder, and a paste.

79. The cosmetic or pharmaceutical composition according to claim 78, wherein the emulsion forms are chosen from oil-in-water (O/W), water-in-oil (W/O) emulsion, and multiple emulsion chosen from W/O/W, polyol/O/W, and O/W/O emulsions.

80. The cosmetic or pharmaceutical composition according to claim 78, wherein the dispersion of vesicles is of ionic or nonionic lipids.

81. The cosmetic or pharmaceutical composition according to claim 62, wherein the pastes are chosen from soft pastes and anhydrous pastes.

82. The cosmetic or pharmaceutical composition according to claim 62, wherein said composition is a hair product.

83. The cosmetic or pharmaceutical composition according to claim 82, wherein the hair product is chosen from lacquers and shampoos.

84. The cosmetic or pharmaceutical composition according to claim 62, wherein said composition is a makeup composition.

85. The cosmetic or pharmaceutical composition according to claim 84, wherein said composition is a nail varnish.

86. A cosmetic process for making up or caring for keratin materials, comprising the application to the keratin materials of a composition comprising a linear block ethylenic copolymer comprising:
at least one block A that is obtained from at least one monomer comprising
a majority amount of an ethylenic monomer comprising a lactam ring chosen from monomers of formula (I): 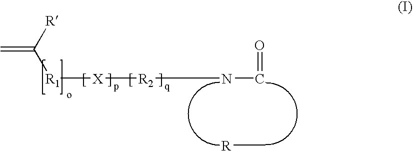
wherein:
R is chosen from —(CH₂)ₙ— radicals, in which at least one carbon atom is optionally replaced with a heteroatom chosen from nitrogen and oxygen atoms and is optionally substituted with at least one C₁ to C₆ alkyl group, and wherein n is an integer ranging from 3 to 12;
R′ is chosen from a hydrogen atom and methyl groups;
R₁ and R₂, which may be identical or different, are chosen from linear, branched and cyclic alkylene and aralkylene groups of 1 to 22 carbon atoms, wherein at least one carbon atom is optionally replaced with a heteroatom chosen from oxygen and nitrogen atoms;
X is chosen from —OCO—, —NHCO—, and —COO— groups and an oxygen atom;
o, p and q represent, independently of each other, 0 or 1; and
a minority amount of at least one non-hydrophilic monomer;
and at least one block B that is obtained from monomers not comprising an ethylenic monomer comprising a lactam ring of formula (I), or comprising a minority proportion thereof.

87. A process for improving the styling power and/or the hold of keratin materials, without having a tacky feel, comprising the application to the keratin materials of a composition comprising a linear block ethylenic copolymer comprising:
at least one block A that is obtained from at least one monomer comprising
a majority amount of an ethylenic monomer comprising a lactam ring chosen from monomers of formula (I): 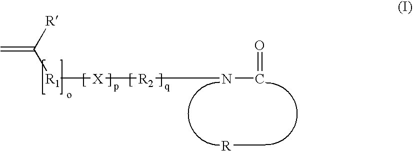
wherein:
R is chosen from —(CH₂)ₙ— radicals, in which at least one carbon atom is optionally replaced with a heteroatom chosen from nitrogen and oxygen atoms and is optionally substituted with at least one C₁ to C₆ alkyl group, and wherein n is an integer ranging from 3 to 12;
R′ is chosen from a hydrogen atom and methyl groups;
R₁ and R₂, which may be identical or different, are chosen from linear, branched and cyclic alkylene and aralkylene groups of 1 to 22 carbon atoms, wherein at least one carbon atom is optionally replaced with a heteroatom chosen from oxygen and nitrogen atoms;
X is chosen from —OCO—, —NHCO—, and —COO— groups and an oxygen atom;
o, p and q represent, independently of each other, 0 or 1; and
a minority amount of at least one non-hydrophilic monomer;
and at least one block B that is obtained from monomers not comprising an ethylenic monomer comprising a lactam ring of formula (I), or comprising a minority proportion thereof.

88. A process for increasing the adhesion and the wear resistance of a nail varnish, without having a tacky feel, comprising combining with the nail varnish a composition comprising a linear block ethylenic copolymer comprising:
at least one block A that is obtained from at least one monomer comprising
a majority amount of an ethylenic monomer comprising a lactam ring chosen from monomers of formula (I): 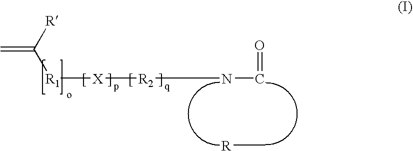
wherein:
R is chosen from —(CH₂)ₙ— radicals, in which at least one carbon atom is optionally replaced with a heteroatom chosen from nitrogen and oxygen atoms and is optionally substituted with at least one C₁ to C₆ alkyl group, and wherein n is an integer ranging from 3 to 12;
R′ is chosen from a hydrogen atom and methyl groups;
R₁ and R₂, which may be identical or different, are chosen from linear, branched and cyclic alkylene and aralkylene groups of 1 to 22 carbon atoms, wherein at least one carbon atom is optionally replaced with a heteroatom chosen from oxygen and nitrogen atoms;
X is chosen from —OCO—, —NHCO—, and —COO— groups and an oxygen atom;
o, p and q represent, independently of each other, 0 or 1; and
a minority amount of at least one non-hydrophilic monomer;
and at least one block B that is obtained from monomers not comprising an ethylenic monomer comprising a lactam ring of formula (I), or comprising a minority proportion thereof.

89. A process for improving the staying power of a makeup composition comprising combining with the makeup composition a composition comprising a linear block ethylenic copolymer comprising:
at least one block A that is obtained from at least one monomer comprising
a majority amount of an ethylenic monomer comprising a lactam ring chosen from monomers of formula (I): 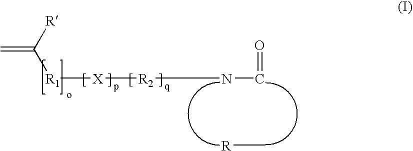
wherein:
R is chosen from —(CH₂)ₙ— radicals, in which at least one carbon atom is optionally replaced with a heteroatom chosen from nitrogen and oxygen atoms and is optionally substituted with at least one C₁ to C₆ alkyl group, and wherein n is an integer ranging from 3 to 12;
R′ is chosen from a hydrogen atom and methyl groups;
R₁ and R₂, which may be identical or different, are chosen from linear, branched and cyclic alkylene and aralkylene groups of 1 to 22 carbon atoms, wherein at least one carbon atom is optionally replaced with a heteroatom chosen from oxygen and nitrogen atoms;
X is chosen from —OCO—, —NHCO—, and —COO— groups and an oxygen atom;
o, p and q represent, independently of each other, 0 or 1; and
a minority amount of at least one non-hydrophilic monomer;
and at least one block B that is obtained from monomers not comprising an ethylenic monomer comprising a lactam ring of formula (I), or comprising a minority proportion thereof.

90. A process for reducing the tack, of a cosmetic composition comprising combining with the cosmetic composition, a composition comprising a linear block ethylenic copolymer comprising:
at least one block A that is obtained from at least one monomer comprising
a majority amount of an ethylenic monomer comprising a lactam ring chosen from monomers of formula (I): 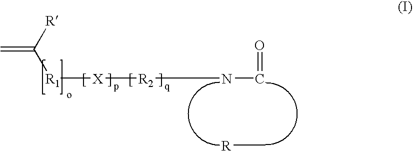
wherein:
R is chosen from —(CH₂)ₙ— radicals, in which at least one carbon atom is optionally replaced with a heteroatom chosen from nitrogen and oxygen atoms and is optionally substituted with at least one C₁ to C₆ alkyl group, and wherein n is an integer ranging from 3 to 12;
R′ is chosen from a hydrogen atom and methyl groups;
R₁ and R₂, which may be identical or different, are chosen from linear, branched and cyclic alkylene and aralkylene groups of 1 to 22 carbon atoms, wherein at least one carbon atom is optionally replaced with a heteroatom chosen from oxygen and nitrogen atoms;
X is chosen from —OCO—, —NHCO—, and —COO— groups and an oxygen atom;
o, p and q represent, independently of each other, 0 or 1; and
a minority amount of at least one non-hydrophilic monomer;
and at least one block B that is obtained from monomers not comprising an ethylenic monomer comprising a lactam ring of formula (I), or comprising a minority proportion thereof.

91. The process according to claim 90, wherein the tack is reduced under conditions of high humidity.

92. The process according to claim 91, wherein the conditions of high humidity range from 50% to 100% RH.

93. A process for giving the skin a smooth appearance or for masking wrinkles comprising the application to the skin and wrinkles of a composition comprising a linear block ethylenic copolymer comprising:
at least one block A that is obtained from at least one monomer comprising
a majority amount of an ethylenic monomer comprising a lactam ring chosen from monomers of formula (I): 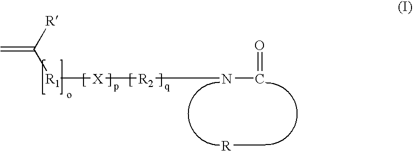
wherein:
R is chosen from —(CH₂)ₙ— radicals, in which at least one carbon atom is optionally replaced with a heteroatom chosen from nitrogen and oxygen atoms and is optionally substituted with at least one C₁ to C₆ alkyl group, and wherein n is an integer ranging from 3 to 12;
R′ is chosen from a hydrogen atom and methyl groups;
R₁ and R₂, which may be identical or different, are chosen from linear, branched and cyclic alkylene and aralkylene groups of 1 to 22 carbon atoms, wherein at least one carbon atom is optionally replaced with a heteroatom chosen from oxygen and nitrogen atoms;
X is chosen from —OCO—, —NHCO—, and —COO— groups and an oxygen atom;
o, p and q represent, independently of each other, 0 or 1; and
a minority amount of at least one non-hydrophilic monomer;
and at least one block B that is obtained from monomers not comprising an ethylenic monomer comprising a lactam ring of formula (I), or comprising a minority proportion thereof.

94. The linear block ethylenic copolymer according to claim 31, wherein the ethylenic hydrocarbons of 2 to 10 carbon atoms are chosen from ethylene, isoprene, and butadiene.

95. The linear block ethylenic copolymer according to claim 31, wherein the halogen atoms are chosen from Cl, Br, I, and F atoms.

96. The linear block ethylenic copolymer according to claim 31, wherein the C₄ to C₃₀ aralkyl groups are chosen from 2-phenylethyl and benzyl groups.

97. The linear block ethylenic copolymer according to claim 31, wherein the heterocyclylalkyl groups are chosen from furfuylmethyl and tetrahydrofurfurylmethyl groups.

98. The linear block ethylenic copolymer according to claim 31, wherein R₃ is chosen from methyl, ethyl, propyl, isobutyl, n-butyl, tert-butyl, hexyl, ethylhexyl, octyl, lauryl, isooctyl, isodecyl, cyclohexyl, t-butylcyclohexyl, t-butylbenzyl, isobornyl, phenyl, furfurylmethyl, tetrahydrofurfurylmethyl, and 2-ethylperfluorohexyl groups, and from (OC₂H₄)ₘ—OR″, wherein m is an integer ranging from 5 to 150 and R″ is chosen from hydrogen atoms and C₆ to C₃₀ alkyl groups.

99. The linear block ethylenic copolymer according to claim 98, wherein R″ is POE-lauryl.

100. The linear block ethylenic copolymer according to claim 31, wherein R₄ and R₅, which may be identical or different are chosen from methyl, ethyl, n-butyl, t-butyl, isopropyl, isohexyl, isooctyl, and isononyl groups.

101. The linear block ethylenic copolymer according to claim 31, wherein the C₃ to C₁₂ cycloalkyl groups are chosen from isobornyl and cyclohexyl groups.

102. The linear block ethylenic copolymer according to claim 31, wherein the C₃ to C₂₀ aryl group is phenyl.

103. The linear block ethylenic copolymer according to claim 31, wherein the (meth)acrylamide monomers are chosen from N-ethyl(meth)acrylamide, N-butylacrylamide, N-t-butylacrylamide, N-isopropylacrylamide, N,N-dimethyl(meth)acrylamide, N-octylacrylamide, N-dodecylacrylamide, and undecylacrylamide.

104. The linear block ethylenic copolymer according to claim 31, wherein the vinyl compounds are vinyl monomers are chosen from vinylcyclohexane and styrene.

105. The linear block ethylenic copolymer according to claim 31, wherein the vinyl compounds are vinyl esters chosen from vinyl acetate, vinyl propionate, vinyl butyrate, vinyl ethylhexanoate, vinyl neononanoate, and vinyl neododecanoate.

106. The linear block ethylenic copolymer according to claim 31, wherein the vinyl compounds are vinyl ethers chosen from ethyl vinyl ether and isobutyl vinyl ether.

107. The linear block ethylenic copolymer according to claim 31, wherein the (meth)acrylic monomer comprising a fluoro or perfluoro group is ethylperfluorooctyl methacrylate.

108. The linear block ethylenic copolymer according to claim 31, wherein the silicone (meth)acrylic monomers are chosen from methacryloxypropyltris(trimethylsiloxy)silane, acryloxypropylpolydimethylsiloxane, and silicone (meth)acrylamides.

109. The linear block ethylenic copolymer according to claim 58, wherein the carboxylic acid salts containing a labile hydrogen are chosen from sodium chloroacetate.

110. The linear block ethylenic copolymer according to claim 58, wherein the cyclic sulfones are chosen from propane sulfone.

111. The linear block ethylenic copolymer according to claim 60, wherein the hydroxyalkyl (meth)acrylates are chosen from hydroxyethyl (meth)acrylate and hydroxypropyl (meth)acrylate.

112. The linear block ethylenic copolymer according to claim 60, wherein the (meth)acrylates and (meth)acrylamides of (C₁-C₄)alkoxy(C₁₋₄)alkyl are chosen from methoxyethyl, ethoxyethyl, and methoxypropyl.

113. The linear block ethylenic copolymer according to claim 60, wherein the (meth)acrylates and (meth)acrylamides comprising a group —(OC₂H₄)ₘ—OR′″ are chosen from POE methoxy and POE-OH.

114. The linear block ethylenic copolymer according to claim 60, wherein the polysaccharide (meth)acrylates are chosen from sucrose acrylate.
